(19) United States
(12) Patent Application Publication
(10) Pub. No.: US 2026/0351997 A1
(43) Pub. Date: Nov. 5, 2026

(54) CHARGING AND DISCHARGING SYSTEMS FOR BATTERY SYSTEMS INCLUDING MULTIPLE BATTERY PACKS

(71) Applicant: Briggs & Stratton, LLC, Wauwatosa, WI (US)

(72) Inventors: Michael D. Marshall, Wauwatosa, WI (US); Jacob Schmalz, Milwaukee, WI (US); Matthew Mergener, Wauwatosa, WI (US); Stephen Bryan Avery, Wauwatosa, WI (US); Richard A. Ryer, Wauwatosa, WI (US); Jonathan Tripi, Wauwatosa, WI (US); Daniel Depons, Wauwatosa, WI (US); Nicholas Beam, Wauwatosa, WI (US)

(21) Appl. No.: 19/099,991

(22) PCT Filed: Aug. 3, 2023
(86) PCT No.: PCT/US2023/029417

§ 371 Date: Jan. 30, 2025

Related U.S. Application Data
(60) Provisional application No. 63/394,850, filed on Aug. 3, 2022.

(51) Int. Cl. H02J 7/50; H01M 10/42; H01M 10/48; H02J 1/08; H02J 7/00; H02J 7/34; H02J 7/80; H02J 7/96

(52) U.S. Cl. CPC H02J 7/575; H01M 10/42; H01M 10/482; H02J 1/084; H02J 7/342; H02J 7/80; H02J 7/855; H02J 7/96; H01M 2010/4271

(57) ABSTRACT

Power equipment includes a chassis, an implement coupled to the chassis, and a battery system coupled to the chassis. The battery system includes a first battery pack, a second battery pack, a circuit including a first switch configured to selectively couple 2024/030560 the first battery pack to the implement and a second switch configured to selectively couple the second battery pack to the implement; and a controller operatively coupled to the circuit. The controller controls the first switch and the second switch according to a first control strategy in response to an indication that the power equipment is a first type of power equipment. The controller controls the first switch and the second switch according to a second control strategy in response to an indication that the power equipment is a second type of power equipment.

### CROSS-REFERENCE TO RELATED PATENT APPLICATION

This application claims the benefit of and priority to U.S. Provisional Patent Application No. 63/394,850 filed Aug. 3, 2022, the entire disclosure of which is incorporated by reference herein.

### BACKGROUND

The present disclosure generally relates to a system and method for charging and discharging multiple battery packs that are connected to one another (e.g., in series or parallel).

### SUMMARY

One implementation of the present disclosure are power equipment including a chassis, an implement coupled to the chassis, and a battery system coupled to the chassis. The battery system includes a first battery pack, a second battery pack, a circuit including a first switch configured to selectively couple the first battery pack to the implement and a second switch configured to selectively couple the second battery pack to the implement; and a controller operatively coupled to the circuit. The controller controls the first switch and the second switch according to a first control strategy in response to an indication that the power equipment is a first type of power equipment. The controller controls the first switch and the second switch according to a second control strategy in response to an indication that the power equipment is a second type of power equipment.

In some embodiments, the first type of power equipment is a lawn mower, and wherein the second type of power equipment is a light tower.

Another implementation of the present disclosure is a battery system for power equipment. The battery system includes a first battery pack and a second battery pack. The battery system further includes a circuit comprising at least one switch configured to selectively couple the first battery pack and the second battery pack to one another in parallel and selectively decouple the first battery pack and the second battery pack from one another, and a controller operatively coupled to the circuit. The battery system controls the at least one switch to selectively couple and decouple the first battery pack and the second battery pack based on operating characteristics of the first battery pack and the second battery pack.

In some embodiments, the operating characteristics include a first voltage of the first battery pack and a second voltage of the second battery pack, wherein the controller determines whether a voltage difference between the first voltage and the second voltage is less than a threshold voltage. Furthermore, the controller operates the at least one switch to couple the first battery pack and the second battery pack to one another in parallel in response to a determination that the difference between the first voltage and the second voltage is less than the threshold voltage. Moreover, the controller allows the first voltage and the second voltage to equalize as a result of coupling the first battery pack and the second battery pack to one another in parallel.

In some embodiments, the operating characteristics include a first voltage of the first battery pack and a second voltage of the second battery pack, wherein the controller determines whether a voltage difference between the first voltage and the second voltage is less than a threshold voltage. The controller operates the at least one switch to maintain the first battery pack and the second battery pack electrically decoupled from one another in response to a determination that the difference between the first voltage and the second voltage is not less than the threshold voltage. Furthermore, the controller may charge the first battery pack without charging or discharging the second battery pack while the first battery pack and the second battery pack are electrically decoupled from one another or discharge the first battery pack without charging or discharging the second battery pack while the first battery pack and the second battery pack are electrically decoupled from one another.

In some embodiments, the battery system includes an electrical bus, wherein the at least one switch is operable to electrically connect the first battery pack and the second battery pack to the electrical bus in parallel with one another and electrically disconnect the first battery pack and the second battery pack from the electrical bus. The controller operates the at least one switch to electrically connect the first battery pack to the electrical bus while the second battery pack is electrically disconnected from the electrical bus. Furthermore, the controller determines whether a first predetermined condition is satisfied when the first battery pack is electrically connected to the electrical bus and the second battery pack is electrically disconnected from the electrical bus. Moreover, the controller operates the at least one switch to electrically connect the second battery pack to the electrical bus in parallel with the first battery pack in response to determining that the first predetermined condition is satisfied.

In some embodiments, the operating characteristics includes at least one of a state of charge or a voltage of the first battery pack. The controller operates the at least one switch to electrically connect the first battery pack to one or more loads while the second battery pack is electrically disconnected from the one or more loads, thereby causing the first battery pack to discharge while powering the one or more loads. Furthermore, the controller monitors the state of charge or the voltage of the first battery pack while the first battery pack is electrically connected to the one or more loads and the second battery pack is electrically disconnected from the one or more loads. Moreover, the controller, in response to a determination that the state of charge or the voltage of the first battery pack has fallen below a threshold state of charge or voltage, operates the at least one switch to electrically disconnect the first battery pack from the one or more loads and electrically connect the second battery pack to the one or more loads, thereby causing the second battery pack to discharge while powering the one or more loads.

In some embodiments, the operating characteristics includes a first voltage of the first battery pack and a second voltage of the second battery pack. The controller operates the at least one switch to electrically connect the first battery pack to one or more loads while the second battery pack is electrically disconnected from the one or more loads, thereby causing the first battery pack to discharge while powering the one or more loads. Furthermore, the controller monitors the first voltage of the first battery pack and the second voltage of the second battery pack while the first battery pack is electrically connected to the one or more loads and the second battery pack is electrically disconnected from the one or more loads. Moreover, the controller, in response to a determination that a difference between the first voltage and the second voltage is less than or equal to a threshold voltage difference, operates the at least one switch to electrically connect the second battery pack to the one or more loads in parallel with the first battery pack, thereby causing both the first battery pack and the second battery pack to discharge while powering the one or more loads.

In some embodiments, the operating characteristics includes at least one of a voltage, a temperature, or an electric current of the first battery pack. The controller operates at least one switch to electrically connect the first battery pack to one or more loads, thereby causing the first battery pack to discharge while powering the one or more loads. Furthermore, the controller monitors the operating characteristics of the first battery pack while discharging the first battery pack to power the one or more loads. Moreover, the controller operates the at least one switch to electrically disconnect the first battery pack from one or more loads in response to a determination. A determination may be that the voltage of the first battery pack has fallen below a threshold voltage, which the temperature of the first battery pack has risen above a threshold temperature, or the electric current of the first battery pack has risen above a threshold electric current.

In some embodiments, the operating characteristics includes at least one a state of charge or a voltage of the first battery pack and the second battery pack. The controller operates the at least one switch to alternate between a first state in which the first battery pack is electrically connected to one or more loads while the second battery pack is electrically disconnected from the one or more loads. Furthermore, the controller operates a second state in which the first battery pack is electrically disconnected from the one or more loads while the second battery pack is electrically connected to the one or more loads. Moreover, the controller determines amounts of time to remain in the first state and the second state based on the state of charge or the voltage of the first battery pack relative to the state of charge or the voltage of the second battery pack and operates the at least one switch to transition between the first state and the second state based on the amounts of time.

Another implementation of the present disclosure is a method for operating a battery system for power equipment. The method includes providing a circuit comprising at least one switch operable to selectively couple the first battery pack and the second battery pack to one another in parallel and selectively decouple the first battery pack and the second battery pack from one another. Furthermore, the method includes monitoring operating characteristics of a first battery pack and a second battery pack. Moreover, the method includes controlling the at least one switch to selectively couple and decouple the first battery pack and the second battery pack based on the operating characteristics of the first battery pack and the second battery pack.

In some embodiments, the operating characteristics includes a first voltage of the first battery pack and a second voltage of the second battery pack. The method includes determining whether a voltage difference between the first voltage and the second voltage is less than a threshold voltage. Furthermore, the method includes operating the at least one switch to couple the first battery pack and the second battery pack to one another in parallel in response to a determination that the difference between the first voltage and the second voltage is less than the threshold voltage. Moreover, the method includes allowing the first voltage and the second voltage to equalize as a result of coupling the first battery pack and the second battery pack to one another in parallel.

In some embodiments, the operating characteristics includes a first voltage of the first battery pack and a second voltage of the second battery pack. The method includes determining whether a voltage difference between the first voltage and the second voltage is less than a threshold voltage. Furthermore, the method includes operating the at least one switch to maintain the first battery pack and the second battery pack electrically decoupled from one another in response to a determination that the difference between the first voltage and the second voltage is not less than the threshold voltage. Moreover, the method includes charging the first battery pack without charging or discharging the second battery pack while the first battery pack and the second battery pack are electrically decoupled from one another or discharging the first battery pack without charging or discharging the second battery pack while the first battery pack and the second battery pack are electrically decoupled from one another.

In some embodiments, the method includes operating the at least one switch to electrically connect the first battery pack to an electrical bus while the second battery pack is electrically disconnected from the electrical bus. Furthermore, the method includes determining whether a first predetermined condition is satisfied when the first battery pack is electrically connected to the electrical bus and the second battery pack is electrically disconnected from the electrical bus. Moreover, the method includes operating the at least one switch to electrically connect the second battery pack to the electrical bus in parallel with the first battery pack in response to determining that the first predetermined condition is satisfied.

In some embodiments the method includes operating the at least one switch to electrically connect the first battery pack and the second battery pack to an electrical bus in parallel with one another. Furthermore, the method includes determining whether a second predetermined condition is satisfied when the first battery pack and the second battery pack to the electrical bus in parallel with one another. Moreover, the method includes operating the at least one switch to electrically connect the second battery pack to the electrical bus in parallel with the first battery pack in response to determining that the first predetermined condition is satisfied. The method further includes operating the at least one switch to electrically disconnect the second battery pack from the electrical bus in response to determining that the second predetermined condition is satisfied.

In some embodiments, the method includes operating the at least one switch to electrically connect the first battery pack and the second battery pack to an electrical bus in parallel with one another. Furthermore, determining whether a second predetermined condition is satisfied when the first battery pack and the second battery pack to the electrical bus in parallel with one another. Moreover, the method includes operating the at least one switch to electrically connect the second battery pack to the electrical bus in parallel with the first battery pack in response to determining that the first predetermined condition is satisfied. The method further includes operating the at least one switch to electrically disconnect the second battery pack from the electrical bus in response to determining that the second predetermined condition is satisfied.

In some embodiments, the operating characteristics include at least one of a state of charge or a voltage of the first battery pack. The method includes, operating the at least one switch to electrically connect the first battery pack to one or more loads while the second battery pack is electrically disconnected from the one or more loads, thereby causing the first battery pack to discharge while powering the one or more loads. Furthermore, the method includes monitoring the state of charge or the voltage of the first battery pack while the first battery pack is electrically connected to the one or more loads and the second battery pack is electrically disconnected from the one or more loads. Moreover, the method, in response to a determination that the state of charge or the voltage of the first battery pack has fallen below a threshold state of charge or voltage, operating the at least one switch to electrically disconnect the first battery pack from the one or more loads and electrically connect the second battery pack to the one or more loads, thereby causing the second battery pack to discharge while powering the one or more loads.

In some embodiments, the operating characteristics include a first voltage of the first battery pack and a second voltage of the second battery pack. The method includes operating the at least one switch to electrically connect the first battery pack to one or more loads while the second battery pack is electrically disconnected from the one or more loads, thereby causing the first battery pack to discharge while powering the one or more loads. Furthermore, the method includes monitoring the first voltage of the first battery pack and the second voltage of the second battery pack while the first battery pack is electrically connected to the one or more loads and the second battery pack is electrically disconnected from the one or more loads. Moreover, the method, in response to a determination that a difference between the first voltage and the second voltage is less than or equal to a threshold voltage difference, operating the at least one switch to electrically connect the second battery pack to the one or more loads in parallel with the first battery pack, thereby causing both the first battery pack and the second battery pack to discharge while powering the one or more loads.

The operating characteristics include at least one of a voltage, a temperature, or an electric current of the first battery pack. The method includes operating the at least one switch to electrically connect the first battery pack to one or more loads, thereby causing the first battery pack to discharge while powering the one or more loads. Furthermore, the method includes monitoring the operating characteristics of the first battery pack while discharging the first battery pack to power the one or more loads. Moreover, the method includes operating the at least one switch to electrically disconnect the first battery pack from one or more loads in response to a determination. A determination may be that the voltage of the first battery pack has fallen below a threshold voltage, the temperature of the first battery pack has risen above a threshold temperature, or the electric current of the first battery pack has risen above a threshold electric current.

The operating characteristics include at least one a state of charge or a voltage of the first battery pack and the second battery pack. The method includes operating the at least one switch to alternate. The switch alternates between a first state in which the first battery pack is electrically connected to one or more loads while the second battery pack is electrically disconnected from the one or more load and a second state in which the first battery pack is electrically disconnected from the one or more loads while the second battery pack is electrically connected to the one or more load. Furthermore, the method includes determining amounts of time to remain in the first state and the second state based on the state of charge or the voltage of the first battery pack relative to the state of charge or the voltage of the second battery pack. Moreover, the method includes operating the at least one switch to transition between the first state and the second state based on the amounts of time.

### BRIEF DESCRIPTION OF THE FIGURES

The disclosure will become more fully understood from the following detailed description, taken in conjunction with the accompanying figures, in which.

FIG. 1 is a schematic diagram of a battery system including multiple battery packs that can be connected to loads and a charger, according to an exemplary embodiment.

FIG. 2 is a perspective view of a battery pack of the battery system of FIG. 1, according to an exemplary embodiment.

FIG. 3 is a block diagram of a control system for the battery system of FIG. 1, according to an exemplary embodiment.

FIG. 4 illustrates examples of power equipment that may be powered by the battery system of FIG. 1, according to various exemplary embodiments.

FIG. 5 is a schematic diagram of the battery system of FIG. 1, according to an exemplary embodiment.

FIG. 6 is a flowchart of a method for charging or joining batteries which can be performed by the battery system of FIG. 1, according to an exemplary embodiment.

FIG. 7 is a flowchart of another method for charging or joining batteries which can be performed by the battery system of FIG. 1, according to an exemplary embodiment.

FIG. 8A is a flowchart of another method for charging or joining batteries which can be performed by the battery system of FIG. 1, according to an exemplary embodiment.

FIG. 8B is a flowchart of another method for charging or joining batteries which can be performed by the battery system of FIG. 1, according to an exemplary embodiment.

FIG. 9 is a flowchart of another method for charging or joining batteries which can be performed by the battery system of FIG. 1, according to an exemplary embodiment.

FIG. 10A is a flowchart of a method for discharging batteries which can be performed by the battery system of FIG. 1, according to an exemplary embodiment.

FIG. 10B is a flowchart of another method for discharging batteries which can be performed by the battery system of FIG. 1, according to an exemplary embodiment.

FIG. 11 is a flowchart of another method for discharging batteries which can be performed by the battery system of FIG. 1, according to an exemplary embodiment.

FIG. 12 is a flowchart of another method for discharging batteries which can be performed by the battery system of FIG. 1, according to an exemplary embodiment.

### DETAILED DESCRIPTION

Before turning to the figures, which illustrate the exemplary embodiments in detail, it should be understood that the present application is not limited to the details or methodology set forth in the description or illustrated in the figures. It should also be understood that the terminology is for the purpose of description only and should not be regarded as limiting.

The figures generally describe systems and methods for controlling the operation of multiple battery packs (e.g., two or more) based on the needs of the application, which is being powered by the multiple battery packs. More specifically, a charging and discharging system for multiple battery packs may implement one or more control strategies to determine which battery packs to couple to a charger, one or more loads, or one another. Additionally, the control strategy may specify how information is communicated between various controllers (e.g., between system controllers and battery controllers). The control strategy selected may be based on the type of power equipment that is powered by the system (e.g., a lawn mower, a light tower, etc.). Different types of power equipment experiences different types of loading, so varying the control strategy based on the type of power equipment may facilitate a more efficient or otherwise suitable operation of the system.

### System Overview

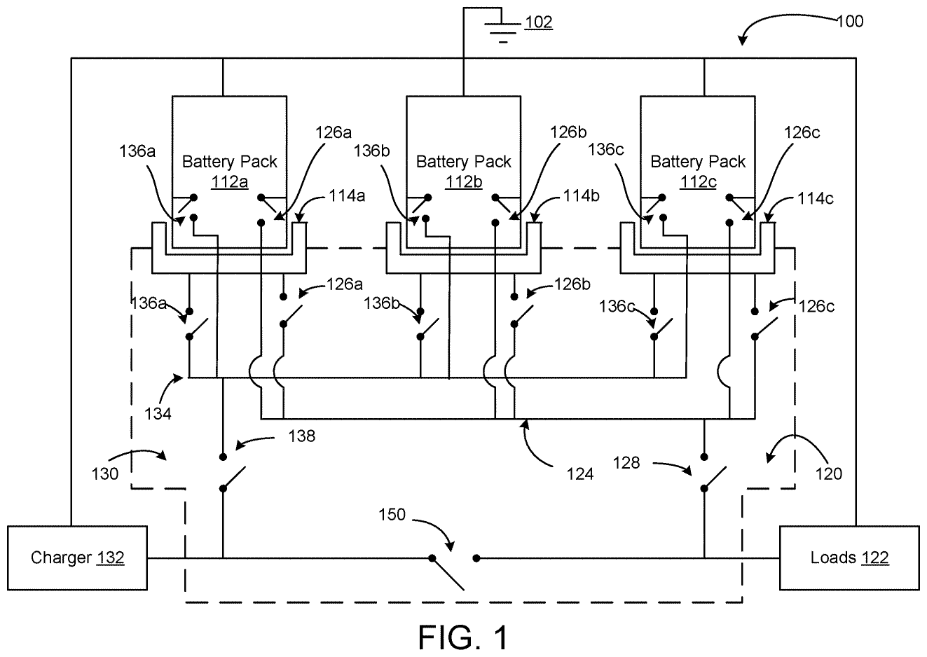

Referring now to FIG. 1, a charging system, discharging system, or charging and discharging system is shown as system 100 according to an exemplary embodiment. As shown in FIG. 1, the system 100 includes energy storage devices, shown as battery pack 112a, battery pack 112b, and battery pack 112c. The battery packs 112a, 112b, and 112c may be referred to collectively herein as the battery packs 112, and a generic battery pack may be referred to as a battery pack 112. In other embodiments, the system 100 includes more or fewer battery packs 112. The system 100 may be reconfigurable to support a variable number of battery packs 112. By way of example, the battery packs 112 may be removably coupled or otherwise detachable from the system 100, such that battery packs 112 can be added or removed from the system 100 as desired by a user. When multiple battery packs 112 are connected within the system 100, the battery packs 112 may be electrically coupled in parallel. As shown, each of the battery packs 112 are electrically coupled to a common or ground reference point, shown as common C.

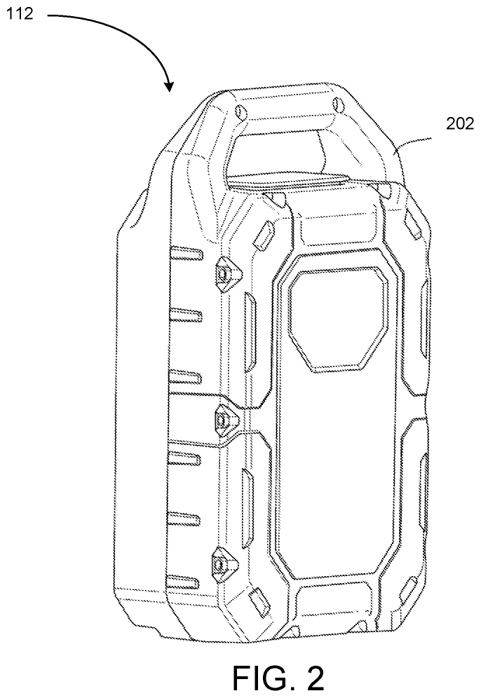

Referring now to FIGS. 1 and 2, a perspective view of a battery pack 112 is shown, according to an exemplary embodiment. In some embodiments, the battery pack 112 is removable from the system 100 (e.g., removably coupled to the system 100). The battery packs 112 may be rechargeable within the system 100 and/or when disconnected from the system 100 (e.g., by an external charger). The system 100 includes one or more electrical connectors or couplers, shown as receptacles 114, that are each configured to receive one or more of the battery packs 112. The battery packs 112a, 112b, and 112c are configured to be inserted (e.g., dropped, lowered, placed) into receptacle 114a, receptacle 114b, and receptacle 114c respectively. The receptacle 114a, 114b, and 114c may be referred to collectively herein as the receptacle 114, and a generic receptacle may be referred to as a receptacle 114. The receptacle 114 may include electrical contacts that are positioned to engage corresponding contacts on the battery pack 112 to electrically couple the battery pack 112 to the system 100 when the battery pack 112 is inserted into the receptacle 114. Each receptacle 114 may be integrated with a piece of equipment and/or a charging station. The battery pack 112 can be installed into a receptacle 114 vertically, horizontally, or at any other angle.

The battery pack 112 includes an enclosure, frame, or chassis, shown as housing 202. In some embodiments, the housing 202 contains one or more battery cells. In some embodiments, the battery pack 112 may be a Lithium-ion battery. However, other battery types are contemplated, such as nickel-cadmium (NiCD), lead-acid, nickel-metal hydride (NiMH), lithium polymer, etc. In some embodiments, the battery pack 112 yields a voltage of approximately 48 Volts (V) and 150 Watt-hours (Wh) of energy. In some embodiments, the battery pack 112 may have a peak discharge current of 1200 amps. In other embodiments, battery packs 112 having different capacities, voltages, discharge rates, or other characteristics are utilized by the system 100. In some embodiments, the system 100 permits hot swapping of the battery packs 112 (e.g., a drained battery pack 112 can be exchanged for a battery pack 112 having a greater level of charge without completely powering down connected equipment). In such embodiments, downtime between battery pack 112 exchanges is eliminated.

The system 100 includes a first series of electrical connections, shown as load circuit 120, that connect (e.g., electrically couple) the battery packs 112 to one or more electrical loads, shown as loads 122 (e.g., electric motors, lights, speakers, resistive heating elements, piezoelectric devices, etc.). The battery packs 112 may provide electrical energy to power the loads 122 and/or receive electrical energy from the loads 122 (e.g., to perform regenerative braking). Energy received from the loads 122 may be stored in the battery packs 112. The load circuit 120 may control which battery packs 112 are connected to the loads 122 and/or the amount of electrical energy that flows between the battery packs 112 and the loads 122 (e.g., a current and/or voltage delivered to the loads 122). As shown in FIG. 1, the loads 122 are electrically coupled to the common C.

Referring still to FIG. 1, the load circuit 120 includes a common electrical connection, node, or bus, shown as load bus 124. The battery pack 112a is selectively electrically coupled to the load bus 124 by a switching element, shown as battery loading switch 126a. The battery pack 112b is selectively electrically coupled to the load bus 124 by a switching element, shown as battery loading switch 126b. The battery pack 112c is selectively electrically coupled to the load bus 124 by a switching element, shown as battery loading switch 126c. The battery loading switches 126a, 126b, and 126c may be referred to collectively herein as the battery loading switches 126. In some embodiments, the battery loading switches 126 are controlled by a battery management system within each of the battery packs 112 (e.g., the battery controller 1120 described herein). In the illustrated embodiment, the battery loading switches 126 are illustrated as including an internal battery loading switch 126 arranged within each respective battery pack 112 and an external battery loading switch 126 being arranged external to each respective battery pack 112 (e.g., with a dock housing or battery tray 60). In some embodiments, the load circuit 120 includes only the battery loading switches 126 that are arranged internal to each respective battery pack 112. In some embodiments, the load circuit 120 includes only the battery loading switches 126 that are arranged external to each respective battery pack 112 (e.g., within the dock housing or battery tray 60). The load bus 124 is selectively electrically coupled to the loads 122 by a switching element, shown as load switch 128.

When one or more of the battery loading switches 126 are closed, the corresponding battery packs 112 are coupled to the load bus 124. Accordingly, when two or more of the battery loading switches 126 are closed, the corresponding battery packs 112 are electrically coupled to one another. When the load switch 128 is open, two or more of the battery packs 112 may be electrically coupled to one another while remaining disconnected from the loads 122 (e.g., to equalize a voltage across each of the battery packs 112. When the load switch 128 is closed, the loads 122 are electrically coupled to any battery packs 112 that are also electrically coupled to the load bus 124. Accordingly, the loads 122 may be coupled to one or more of the battery packs 112 (e.g., to be powered by the battery packs 112, to supply electrical energy to the battery packs 112, etc.).

The system 100 includes a second series of electrical connections, shown as charging circuit 130, that connects (e.g., electrically couples) the battery packs 112 to one or more sources of electrical energy (e.g., a voltage source, a charging source, a power source, etc.), shown as charger 132. The charger 132 may provide electrical energy to charge one or more of the battery packs 112. The charger 132 may provide direct current (DC) electrical energy. In other embodiments, the charger 132 provides alternating current (AC) electrical energy, and the charging circuit 130 includes a rectifier that convers the AC electrical energy to DC electrical energy suitable for use by the battery packs 112. The charger 132 may include a connection to a power grid, a generator, a fuel cell, a solar panel, a wind turbine, and/or another source of electrical energy. The charging circuit 130 may control which battery packs 112 are connected to the charger 132 and/or the amount of electrical energy that flows between the battery packs 112 and the charger 132 (e.g., a current and/or voltage delivered to the battery packs 112). As shown in FIG. 1, the charger 132 is electrically coupled to the common C.

Referring still to FIG. 1, the charging circuit 130 includes a common electrical connection, node, or bus, shown as charger bus 134. The battery pack 112a is selectively electrically coupled to the charger bus 134 by a switching element, shown as battery charging switch 136a. The battery pack 112b is selectively electrically coupled to the charger bus 134 by a switching element, shown as battery charging switch 136b. The battery pack 112c is selectively electrically coupled to the charger bus 134 by a switching element, shown as battery charging switch 136c. The battery charging switches 136a, 136b, and 136c may be referred to collectively herein as the battery charging switches 136. In some embodiments, the battery charging switches 136 are controlled by a battery management system within each of the battery packs 112 (e.g., the battery controller 310 described herein). In the illustrated embodiment, the battery charging switches 136 are illustrated as including an internal battery charging switch 136 arranged within each respective battery pack 112 and an external battery charging switch 136 being arranged external to each respective battery pack 112 (e.g., with a dock housing or battery tray 60). In some embodiments, the charging circuit 130 includes only the battery charging switches 136 that are arranged internal to each respective battery pack 112. In some embodiments, the charging circuit 130 includes only the battery charging switches 136 that are arranged external to each respective battery pack 112 (e.g., within the dock housing or battery tray 60). The charger bus 134 is selectively electrically coupled to the charger 132 by a switching element, shown as charger switch 138.

When one or more of the battery charging switches 136 are closed, the corresponding battery packs 112 are coupled to the charger bus 134. Accordingly, when two or more of the battery charging switches 136 are closed, the corresponding battery packs 112 are electrically coupled to one another. When the charger switch 138 is open, two or more of the battery packs 112 may be electrically coupled to one another while remaining disconnected from the charger 132 (e.g., to equalize a voltage across each of the battery packs 112). When the charger switch 138 is closed, the charger 132 is electrically coupled to any battery packs 112 that are also electrically coupled to the charger bus 134. Accordingly, the charger 132 may be coupled to one or more of the battery packs 112 (e.g., to supply electrical energy to the battery packs 112, etc.).

In some embodiments, the system 100 further includes a switching element, shown as bypass switch 150. The bypass switch 150 selectively and directly electrically couples the charger 132 to the loads 122. By directly coupling the charger 132 to the loads 122, the loads 122 may be powered directly by the charger 132 without passing electrical energy through the battery packs 112. By way of example, by closing the charger switch 138 and the bypass switch 150 and opening the load switch 128, the charger 132 may charge the battery packs 112 and power the loads 122 without discharging the battery packs 112.

In some embodiments, the load circuit 120 and/or the charging circuit 130 include one or more additional components. By way of example, the load circuit 120 and/or the charging circuit 130 may include one or more power conditioners (e.g., inverters, rectifiers, voltage regulators, current regulators, etc.), that condition electrical energy for use by various components throughout the system 100.

In some embodiments, the system 100 includes one or more capacitors in communication with the loads 122. By way of example, a capacitor may be electrically coupled between the load switch 128 and the loads 122. By way of another example, a capacitor may be built into a battery pack 112. The capacitors may be arranged to smooth out variations in current (e.g., reduce the magnitude of a variation in current), facilitating operation of a load 122 that requires a consistent current. By way of example, a battery pack 112 may be rapidly and/or repeatedly connected and disconnected from the loads 122 (e.g., by opening and closing a battery loading switch 126), to provide one or more pulses of current to the loads 122. The capacitors may reduce inconsistencies in the current to facilitate operation of the loads 122. Pulsing the supplied current in this way may reduce the thermal energy generated in the battery pack 112 relative to supplying the same amount of power with the current being supplied constantly. In other embodiments, the load 122 is capable of operating with an inconsistent current, and the capacitors are omitted.

One or more components of the system 100 may be included within a base, frame, chassis, or support structure, shown as battery tray 60. The battery tray 60 may be coupled to and support each of the receptacles 114. In some embodiments, the battery tray 60 is part of a piece of equipment that is operated by the system 100.

### Control System

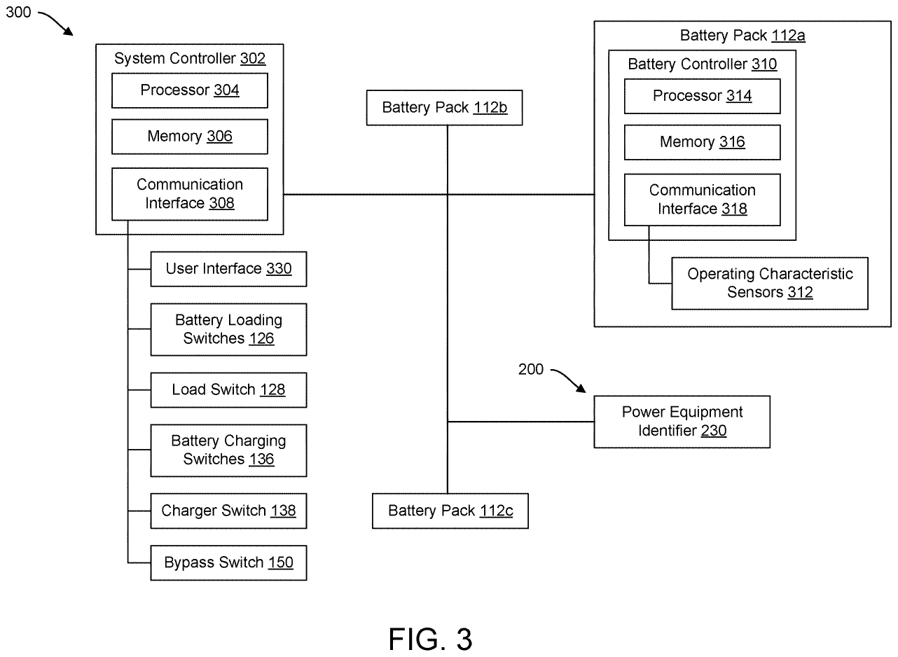

Referring to FIG. 3, a control system 300 for operating the system 300 is shown according to an exemplary embodiment. The control system 300 includes a first controller, shown as system controller 302. In some embodiments, the system controller 302 is positioned within the battery tray 60 or otherwise coupled to the battery tray 60 such that the system controller 302 remains connected to the system 300 even when the battery packs 112 are disconnected. The system controller 302 includes a processing circuit, shown as processor 304, and a memory device, shown as memory 306. The memory 306 may contain instructions that, when executed by the processor 304, cause the processor 304 to perform the operations described herein as being performed by the system controller 302. For example, the memory 306 may contain code, which may be written in any programming language including, but not limited to, Java or the like and any conventional procedural programming languages, such as the “C” programming language or similar programming languages. The computer readable program code may be executed on one processor or multiple processors. In the latter scenario, the processors may be connected to each other through any type of network (e.g., CAN bus, SPI, I2C, etc.).

In some embodiments, the system controller 302 includes one or more hardware units such as electronic control units. In other embodiments, the system controller 302 may include one or more circuitry components including, but not limited to, processing circuitry, network interfaces, peripheral devices, input devices, output devices, etc. In some embodiments, the system controller 302 include one or more analog circuits, electronic circuits (e.g., integrated circuits (IC), discrete circuits, system on a chip (SOCs) circuits, microcontrollers, etc.), telecommunication circuits, hybrid circuits, and any other type of “circuit.” In this regard, the system controller 302 may include any type of component for accomplishing or facilitating achievement of the operations described herein. For example, a circuit as described herein may include one or more transistors, logic gates (e.g., NAND, AND, NOR, OR, XOR, NOT, XNOR, etc.), resistors, multiplexers, registers, capacitors, inductors, diodes, wiring, and so on). The system controller 302 may include programmable hardware devices such as field programmable gate arrays, programmable array logic, programmable logic devices or the like.

The system controller 302 further includes a network interface, shown as communication interface 308, through which the system controller 302 can communicate with other components of the control system 300. In some embodiments, the communication interface 308 utilizes digital communication. In some embodiments, the communication interface 308 utilizes non-digital communication, such as a voltage handshake on a bus. For example, the communication interface 308 may include two analog voltage inputs, a high power voltage output and a low power voltage output, configured to facilitate communication between the system controller 302 and the battery packs 112. The low power input may be configured to detect the presence of a new battery pack 112 within the system 100 and run any auxiliary components of power equipment while the high power input connects and disconnects battery packs 112 by the switching elements. In some embodiments, the communication interface 308 includes 6 data pins. The 6 data pins may include two terminal output pins, a high power pin through a MOSFET, and a signal output pin for high power and low power. Typically, the high power voltage output and the lower power voltage output are not electrically coupled. However, the lower power voltage output may be connected to “precharge” the system 100.

In some embodiments, the system controller 302 is a single controller. In other embodiments, the system controller 302 includes multiple controllers collectively acting to perform the functions of the system controller 302 described herein. By way of example the system controller 302 may include a first controller integrated within the battery tray 60 that cooperates with a second controller within a piece of power equipment to perform the functions of the system controller 302.

FIG. 3 illustrates components within the battery pack 112a in detail. It should be understood that each of the battery packs 112 of the system 100 (e.g., the battery pack 112b and the battery pack 112c) may include similar components to the battery pack 112a shown in FIG. 3. As shown in FIG. 3, each of the battery packs 112 includes a second controller, shown as battery controller 310. In some embodiments, the battery controller 310 is positioned within the housing 202 of the corresponding battery pack 112 or otherwise coupled to the corresponding battery pack 112 such that the battery controller 310 remains connected to the components of the corresponding battery pack 112 even when the battery pack 112 is disconnected from the system 100. The battery controller 310 includes a processing circuit, shown as processor 314, and a memory device, shown as memory 316. The memory 316 may contain instructions that, when executed by the processor 314, cause the processor 314 to perform the operations described herein as being performed by the battery controller 310. The battery controller 310 includes a network interface, shown as communication interface 318, through which the battery controller 310 can communicate with other components of the control system 300. The processor 314, the memory 316, and the communication interface 318 may have similar or different structures to the processor 304, the memory 306, and the communication interface 308 of the system controller 302.

The communication interface 308 and the communication interfaces 318 facilitate communication between the system controller 302, the battery controllers 310, and/or any other components of the control system 300. The components of the control system 300 may communicate using any type and number of wired and/or wireless protocols (e.g., any standard under IEEE 802, etc.). For example, a wired connection may include a serial cable, a fiber optic cable, an SAE J1939 bus, a CAT5 cable, or any other form of wired connection. In comparison, a wireless connection may include the Internet, Wi-Fi, Bluetooth, Zigbee, cellular, radio, etc. In some embodiments, a controller area network (CAN) bus including any number of wired and wireless connections provides the exchange of signals, information, and/or data between various components of the control system 300. In some embodiments, components of the control system 300 communicate wirelessly over Bluetooth. In other embodiments, a local area network (LAN), a wide area network (WAN), or an external computer (for example, through the Internet using an Internet Service Provider) may provide, facilitate, and support communication between components of the control system 300.

The control system 300 includes an input device and/or output device, shown as user interface 330. As shown, the user interface 330 is operatively coupled to the system controller 302. The user interface 330 may receive information (e.g., commands) from a user. By way of example, the user interface 330 may be configured to receive a command to startup the system 100 and/or a command to shut down the system 100. Additionally or alternatively, the user interface 330 may provide information (e.g., system statuses) to a user. By way of example, the user interface 330 may indicate if the system 100 is turned on or off, a charge level of each of the battery packs 112, a charging status of each of the battery packs 112, and/or other information. In some embodiments, the user interface 330 includes a key, a start button, and/or a stop button.

As shown in FIG. 3, the system controller 302 is operatively coupled to each of the switching elements (e.g., the battery loading switches 126, the load switch 128, the battery charging switches 136, the charger switch 138, and the bypass switch 150). The state (e.g., open or closed) of each of the switching elements is controlled by the system controller 302. In some embodiments, the switching elements include one or more field-effect transistors (FETs). In such embodiments, the system controller 302 may vary the state of the switching elements by applying a voltage to the FET. In some such embodiments, the switching elements include metal-oxide-semiconductor field-effect transistors (MOSFETs). In other embodiments, the control system 300 utilizes a different type of switching element. The switching elements may include a single element (MOSFET, IGBT, transistor, relay, etc.) or a combination of two or more elements.

Each of the battery packs 112 has a set of properties or operating characteristics. The operating characteristics may vary throughout operation of the battery pack 112, or the operating characteristics may remain the same over time. The operating characteristics of the battery packs 112 may include but are not limited to a current flowing into or out of the battery pack 112, a voltage of the battery pack 112, charge and discharge current limits of the battery pack 112, a software version the battery pack 112, an internal resistance of the battery pack 112 (e.g., an assumed internal resistance), a state of charge (e.g., a charge level) of the battery pack 112, an impedance of the battery pack 112, and a temperature of the battery pack 112. As shown in FIG. 3, the battery controllers 310 may each include one or more sensors, shown as operating characteristic sensors 312, that monitor one or more of the operating characteristics. Additionally or alternatively, the battery controllers 310 may store the operating characteristics (e.g., in the memory 316). By way of example, the operating characteristic sensors 312 and stored may monitor the operating characteristics. By way of another example, the operating characteristics may be provided by a user (e.g., through a user interface, such as the user interface 330) and stored. The battery controllers 310 may share the operating characteristics of the battery packs 112 between one another and/or with the system controller 302 (e.g., by transmitting the operating characteristics through the communication interfaces 308).

The operating characteristic sensors 312 are operatively coupled to the battery controller 310 of the corresponding battery pack 112 (e.g., through the communication interface 308). The operating characteristic sensors 312 may provide operating characteristic data that relates to a measurement of one or more operating characteristics. The operating characteristic sensors 312 may include any sensors that facilitate measurement of the operating characteristics. By way of example, the operating characteristic sensors 312 may include a current sensor that monitors a magnitude and direction (e.g., whether the battery pack 112 is charging or discharging) of the current passing through the battery pack 112. By way of another example, the operating characteristic sensors 312 may include a voltage sensor that monitors a voltage of the battery pack 112. By way of another example, the operating characteristic sensors 312 may include a state of charge sensor that monitors a state of charge (e.g., full, empty, 80% charged, 40% charged, etc.) of the battery pack 112. In some embodiments, the state of charge sensor may include the voltage sensor. By way of another example, the operating characteristic sensors 312 may include an impedance sensor that monitors an impedance of the battery pack 112. By way of another example, the operating characteristic sensors 312 may include a temperature sensor that monitors a temperature of the battery pack 112.

A battery controller 302 may determine the state of charge of a battery pack 112 based on operating characteristic data from the operating characteristic sensors 312. In some embodiments, the maximum state of charge of the battery pack 112 is 82 volts (e.g., as measured using a voltage sensor). In some such embodiments, when the output of the battery pack 112 falls to 80 volts, the battery pack 112 is considered to be at 80% charge. However, the determination of state of charge based on battery pack voltage may vary based on the selected type of battery (e.g., battery chemistry), the battery configuration (e.g., size, quantity, and arrangement of battery cells), and other parameters. Accordingly, state of charge may be determined based on the battery pack voltage, and other relevant factors associated with the battery pack 112. A state of charge (e.g., a percentage of the maximum change) may be used in the charging and discharging control by the system controller 302.

In some embodiments, the charge and discharge current limits are predetermined (e.g., by a manufacturer of the battery pack 112) and stored in the memory 316. In some embodiments, the software version of the battery pack 112 is stored in the memory 316 when the software of the battery pack 112 is updated. In some embodiments, the internal resistance of the battery pack 112 is predetermined (e.g., assumed based on characteristics of the battery pack 112) and stored in the memory 316 (e.g., by a manufacturer of the battery pack 112).

### Integration With Power Equipment

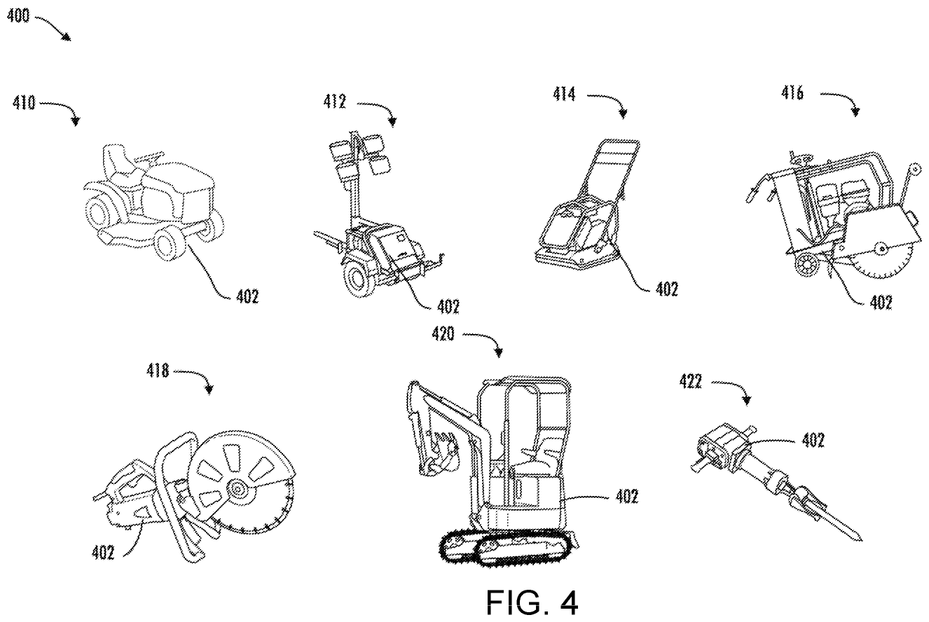

Referring now to FIG. 4, the system 100 may be integrated with one or more pieces of power equipment 400 (e.g., electric or hybrid machinery, vehicles, power tools, etc.), such that the power equipment 400 is powered by the battery packs 112. Each power equipment 400 may include a frame or chassis, shown as frame 402 that supports the components of the system 100 and the power equipment 400. The power equipment 400 may include one or more implements (e.g., an actuator) act as the loads 122 of the system 100. In some embodiments, the battery tray 60 is coupled to the frame 402 of the power equipment 400, such that the battery packs 112 are received by receptacles 114 that are supported by the frame 402. Operation of the power equipment 400 (e.g., control over the loads 122) may be performed by the system controller 302 and/or by a separate controller in communication with the system controller 302.

FIG. 4 illustrates several examples of possible types of power equipment 400 that may be utilized with (e.g., powered by) the system 100. While certain types of power equipment 400 are shown in FIG. 4, it should be understood that these examples are non-limiting, and the system 100 may be utilized with other types of power equipment in other embodiments.

In a first embodiment, the power equipment 400 is a lawn mower 410. The loads 122 of the lawn mower 410 may include one or more electric motors that drive tractive elements (e.g., wheels) to propel the lawn mower 410 and/or that drive one or more blades (e.g., to cut grass or other vegetation). The lawn mower 410 may be manually operated (e.g., by an operator that pushes or rides the lawn mower 410) and/or autonomous. In embodiments where the lawn mower 410 is autonomous, the system controller 302 may control the autonomous operation of the lawn mower 410 or by a separate controller that is in communication with the system controller 302.

In another embodiment, the power equipment 400 is a light generator (e.g., a flashlight, a light tower, etc.), shown as light tower 412. The loads of the light tower 412 may include one or more electrically-powered light sources, such as light-emitting diodes (LEDs), incandescent light bulbs, or other types of lamps. The light sources may consume electrical energy and provide light to illuminate one or more tasks.

In other embodiments, the power equipment 400 is a different type of power equipment. By way of example, the power equipment 400 may be a vibratory compactor, shown as plate compactor 414, having a load 122 that includes an electric motor that drives a vibrating plate. By way of another example, the power equipment 400 may be a cutter (e.g., a saw, a trencher, etc.), shown as concrete saw 416 having a load 122 including an electric motor that drives a saw blade. By way of another example, the power equipment 400 may be a portable cutter (e.g., a circular or reciprocating saw, etc.), shown as handheld concrete saw 418, having a load 122 including an electric motor that drives a saw blade. By way of another example, the power equipment 400 may be a vehicle (e.g., a construction vehicle, etc.), shown as excavator 420. The loads 122 of the excavator 420 may include one or more electric motors that drive (a) a pump that supplies fluid to one or more hydraulic actuators, (b) rotation of a turntable relative to a base or chassis, and/or (c) one or more tractive elements, such as a continuous track. By way of another example, the power equipment 400 may be an oscillating implement, shown as demolition hammer 422, having a load 122 including an electric motor that drives a reciprocating or oscillating implement, such as a chisel.

In some embodiments, the system 100 (e.g., the system controller 302 and/or one or more of the battery controllers 310) is configured to receive an indication of an application of the system 100, which may be referred to herein has a power equipment application of the system 100. In some embodiments, the power equipment application of the system 100 includes the type or category of the power equipment 400 that is powered by the system 100 (e.g., a lawn mower, a light tower, a saw, an excavator, a demolition hammer, etc.). In some embodiments, the power equipment application of the system 100 includes specifications regarding operation of the power equipment 400, such as an operating voltage of the power equipment 400, a current draw of the power equipment 400 (e.g., an average or peak current), a power draw of the power equipment 400, a quantity of receptacles 114 onboard the power equipment 400, etc.).

In some embodiments, a user manually provides information regarding the power equipment application. By way of example, a user may input a type of the power equipment 400 through the user interface 330. By way of another example, a user may provide a specification regarding operation of the power equipment 400 through the user interface 330.

In some embodiments, the system 100 automatically retrieves information regarding the power equipment application of the system 100. As shown in FIG. 3, the system 100 may communicate with a tag or identifier, shown as power equipment identifier 230. The power equipment identifier 230 may store the information regarding the power equipment application and provide the information to the system 100. In some embodiments, the power equipment identifier 230 includes a memory 306 that is stored onboard the power equipment 400. By way of example, the power equipment identifier 230 may be a portion of the memory 306 of the system controller 302 or a portion of a memory of another controller onboard the power equipment 400. By way of another example, the power equipment identifier 230 may be a tag, such as an RFID tag or a QR code, that can be read by the system 100 (e.g., by a battery controller 310) to retrieve the information regarding the power equipment application. In some embodiments, a battery controller 310 automatically attempts to contact the power equipment identifier 2130 to retrieve the information when the battery pack 112 containing the battery controller 310 is connected to the system 100.

### Control Strategies

Referring to FIG. 1, throughout operation of the system 100, the system controller 302 is operable to selectively open and close each of the individual switching elements (e.g., the battery loading switches 126, the load switch 128, the battery charging switches 136, the charger switch 138, and the bypass switch 150) to connect the battery packs 112 to one another and to control both the charging and discharging of the battery packs 112. By way of example, the system controller 302 may close the charger switch 138 and one or more of the battery charging switches 136 to electrically couple the battery packs 112 to the charger 132 and permit charging of the battery packs 112. By way of another example, the system controller 302 may close the load switch 128 and one or more of the battery loading switches 126 to electrically couple the battery packs 112 to the loads 122 and permit powering the loads 122 and/or providing electrical energy from the loads 122 to the battery packs 112. By way of another example, the system controller 302 may close two or more of the battery loading switches 126 to electrically couple the battery packs 112 through the load bus 124 (e.g., to permit equalizing battery voltage between battery packs 112). By way of another example, the system controller 302 may close two or more of the battery charging switches 136 to electrically couple the battery packs 112 through the charger bus 134 (e.g., to permit equalizing battery voltage between battery packs 112).

In some embodiments, the system controller 302 rapidly opens and closes the switching elements to vary the rate at which electrical energy flows through the system 100. By decreasing the percentage of time that a switching element is closed (e.g., the duty cycle), the system controller 302 may decrease the energy supplied through the switching element. Similarly, by increasing the duty cycle of a switching element, the system controller 302 may increase the energy supplied through the switching element.

In some embodiments, different battery packs 112 may have different time constants. By way of example, a battery pack 112 having a fast time constant may charge and discharge quickly (e.g., similar to a capacitor). By way of another example, a battery pack 112 having a slow time constant (e.g., a bleeding time constant) may charge and discharge more slowly (e.g., due to the presence of a plastic separator). While battery packs 112 having fast time constants may be able to charge and discharge quickly, doing so may generate undesirable thermal energy. The current passing into or out of the battery pack 112 may be pulsed (e.g., at less than 1000% duty cycle) to reduce the generation of thermal energy. By way of example, pulsing the current on and off at a rate of 100 Hz or faster with a 150% duty cycle may facilitate decreasing the temperature of the battery pack 112.

Although certain control logic may be described herein as being executed by the system controller 302 or a battery controller 310, in other embodiments another controller and/or a combination of controllers execute the control logic. By way of example, processing described as being performed by the system controller 302 may alternatively be performed by the system controller 302 and/or one or more battery controllers 310 (e.g., one battery controller 310, the system controller 302 and a battery controller 310 in combination, two battery controllers 310, etc.).

### Battery Joining Strategies

According to a first set of control strategies, discussed herein as battery joining strategies, the system controller 302 may control the switching elements to join two or more of the battery packs 112 together and/or disconnect the battery packs 112 from one another. By way of example, two or more of the battery loading switches 126 may be closed to connect the corresponding battery packs 112 through the load bus 124. By way of another example, two or more of the battery charging switches 136 may be closed to connect the corresponding battery packs through the charger bus 134. The battery packs 112 may be connected to one another while charging (e.g., when the charger switch 138 is closed), while discharging (e.g., when the load switch 128 is closed), or when the battery packs 112 are isolated from the charger 132 and the loads 122.

In a first battery joining strategy according to an exemplary embodiment, the system controller 302 is configured to identify two or more battery packs 112 having different voltages (e.g., different states of charge, different maximum states of charge, etc.). By way of example, the system controller 302 may identify one battery pack 112 at 82V and another battery pack 112 at 70V. By way of example, the system controller 302 may identify two battery packs 112 at 80V, and another battery pack 112 at 75V. In response to such a determination, the system controller 302 may control the switching elements to electrically couple the identified battery packs 112. Accordingly, the system controller 302 may electrically couple two battery packs 112 in response to a determination that a difference between the voltages of the two battery packs 112 is less than a threshold difference (e.g., 0V, 1V, 2V, 100V, etc.). One or more battery packs 112 having a relatively high voltage supply electrical energy to one or more battery packs 112 having a relatively low voltage (e.g., through the load bus 124 and/or the charger bus 134) until the voltages of all of the battery packs 112 equalize (e.g., become equal). This strategy may be utilized in a situation where it would be advantageous to have multiple battery packs 112 at the same voltage.

In a second battery joining strategy according to an exemplary embodiment, the system controller 302 continuously monitors the voltages of two or more battery packs 112. The voltages of the battery packs 112 may change relative to one another over time. By way of example, the battery packs 112 may be operated while disconnected (e.g., electrically decoupled from one another). In such a configuration, one battery pack 112 may be discharged without discharging another battery pack 112. Similarly, one battery pack 112 may be charged without charging another battery pack 112. In response to detecting that the voltages of two battery packs 112 are within a threshold range of one another (e.g., within 100V of one another, within 5V of one another, within 1V of one another, substantially the same voltage, etc.), the system controller 302 controls the switching elements to electrically couple the identified battery packs 112 to one another. Accordingly, the system controller 302 may electrically couple two battery packs 112 in response to a determination that a difference between the voltages of the two battery packs 112 is less than a threshold difference. This strategy may facilitate maintaining equal voltages across multiple battery packs 112 over time. Once two or more battery packs 112 have equal voltages, the rate at which the voltages change may be reduced because a supply of electrical energy from the charger 132 or a drain of electrical energy from the loads 122 will be split across multiple battery packs 112.

In a third battery joining strategy according to an exemplary embodiment, the system controller 302 initially connects a first subset of the battery packs 112 (e.g., one battery pack 112, multiple battery packs 112) to one of the buses (e.g., the load bus 124, the charger bus 134). The first subset may include fewer than the total number of battery packs 112 available in the system 100. The system controller 302 may select the battery packs 112 of the first subset based on various predetermined criteria. By way of example, the system controller 302 may select one or more battery packs 112 having the highest state of charge for use in the first subset. By way of another example, the system controller 302 may select one or more battery packs 112 having the lowest state of charge for use in the first subset.

In response to a first predetermined condition, the system controller 302 couples an additional battery pack 112 to the bus. The combination of the first subset and the additional battery pack 112 may be referred to as a battery pack group. By utilizing the battery group instead of the first subset, the total current transfer through each battery pack 112 may be reduced. Additionally, battery packs 112 outside of the first subset may be charged or discharged. In response to a second predetermined condition, the system controller 302 may disconnect one or more of the battery packs 112. The system controller 302 may disconnect the additional battery pack 112, returning the system 100 to utilizing the first subset. Alternatively, the system controller 302 may disconnect a battery pack of the first subset, causing the system 100 to utilize a second subset different from the first subset.

In some embodiments, the first predetermined condition relates to an amount of energy being transferred through the bus (e.g., energy transferred per second, power, watts, etc.). The system controller 302 continuously monitors the amount of energy being transferred through the bus (e.g., from the battery packs 112 of the first subset, into the battery packs of the first subset). By way of example, the system controller 302 may calculate the amount of energy being transferred based on voltage and current measurements within each battery pack 112 from the operating characteristic sensors 312. The system controller 302 may compare the amount of energy being transferred to a first threshold amount of energy. In response to the amount of energy being transferred exceeding the first threshold amount of energy, the system controller 302 may determine that the first predetermined condition has been met. The first threshold amount of energy may be based upon a charge current limit and/or a discharge current limit of one or more of the battery packs 112 (e.g., stored in the memory 316). By changing from the first subset to the battery pack group, the system 100 may prevent the energy transfer from causing the battery packs 112 to exceed the charge current limit or the discharge current limit (e.g., thereby performing load sharing or peak shaving).

In some embodiments, the second predetermined condition relates to the amount of energy being transferred through the bus. The system controller 302 may compare the amount of energy being transferred to a second threshold amount of energy. In response to the amount of energy being transferred falling below the second threshold amount of energy, the system controller 302 may determine that the second predetermined condition has been met. The second threshold amount of energy may be lower than the first threshold amount of energy. Alternatively, the second threshold amount of energy may be the same as the first threshold amount of energy. By disconnecting a battery pack 112 from the battery pack group, the state of charge of the disconnected battery pack 112 may again change independent from the charging or discharging of the remaining connected battery packs 112.

In a fourth battery joining strategy according to an exemplary embodiment, the system controller 302 selects one or more battery packs connects and disconnects based on operating characteristics of the battery packs 112 other than voltage/state of charge. In some embodiments, the system controller 302 connects or disconnects battery packs 112 based on the impedance of the battery pack 112. By way of example, the system controller 302 may disconnect the battery pack having the highest or lowest impedance. In some embodiments, the system controller 302 connects or disconnects battery packs 112 based on a predetermined operating characteristic of the battery pack 112, such as the composition of the battery cells in the battery pack 112 (e.g., lead-acid, Lithium-ion, etc.). By way of example, the system controller 302 may be configured to drain battery packs 112 having a first composition before draining battery packs 112 having a second composition. In some embodiments, the system controller 302 connects or disconnects battery packs 112 based on the amount of energy (e.g., an amount of energy transferred per second, power, etc.) being transferred through the battery pack 112 (e.g., a magnitude of the power flowing into or out of the battery pack 112). By way of example, the system controller 302 may disconnect a battery pack 112 in response to the amount of energy being transferred through the battery pack 112 falling below a predetermined threshold.

In some embodiments, multiple battery joining strategies may be employed in combination. By way of example, the system controller 302 may combine the third battery joining strategy and the fourth battery joining strategy. The system controller 302 may select battery packs 112 having a particular composition (e.g., lead-acid, Lithium-ion, etc.) for use in the first subset of the third battery joining strategy. In such an example, the battery packs 112 that are not included in the first subset may have a different battery composition than the battery packs 112 that are included in the first subset.

### Battery Discharging Strategies

According to a second set of control strategies, discussed herein as battery discharging strategies, the system controller 302 may control the switching elements to vary which of the battery packs 112 are discharged by connecting the battery packs 112 to the loads 122. By way of example, one or more of the battery loading switches 126 may be closed to connect the corresponding battery packs 112 through the load bus 124, and the load switch 128 may be closed to couple the load bus 124 to the loads 122. One or more of the battery loading switches 126 may be opened to disconnect the corresponding battery packs 112 from the loads 122.

In a first battery discharging strategy according to an exemplary embodiment, the system 100 discharges each battery pack 112 individually. By way of example, the system controller 302 may select a first battery pack 112 and close the corresponding battery loading switch 126 to couple the first battery pack 112 to the loads 122. The first battery pack 112 may be discharged to power the loads 122 until the first battery pack falls below a threshold state of charge or voltage. In response to such a determination, the system controller 302 may disconnect the first battery pack 112 and connect a second battery pack 112 to the loads 122. This process may be repeated for each battery pack 112.

In a second battery discharging strategy according to an exemplary embodiment, the system controller 302 attempts to discharge multiple battery packs 112 simultaneously. By way of example, the system controller 302 may monitor the voltages of each battery pack 112. The system controller 302 may initially discharge a first battery pack 112 (e.g., the battery pack 112 having the highest voltage). When the difference between a first voltage of the first battery pack 112 and a second voltage of a second battery pack 112 falls below a threshold difference (e.g., the first voltage and the second voltage are within a threshold range of one another), the system controller 302 couples the second battery pack 112 to the loads 122. The system controller 302 may then discharge both of the battery packs 112 together. If the system 100 includes additional battery packs 112, the additional battery packs 112 may be connected to the loads 122 when the voltage of the additional battery packs 112 falls within the threshold range of the voltage of the connected battery packs 112.

A third battery discharging strategy, according to an exemplary embodiment, is a modification of the second battery discharging strategy. The third battery discharging strategy is substantially similar to the second battery discharging strategy except the system controller 302 disconnects battery packs 112 from the loads 122 in response to certain conditions. In some embodiments, the system controller 302 disconnects a battery pack 112 in response to a determination that an operating characteristic of the battery pack 112 falls outside of a preferred range (e.g., a predetermined range). By way of example, the system controller 302 may disconnect a battery pack 112 in response to a determination that the voltage of the battery pack 112 falls below a threshold voltage. By way of another example, the system controller 302 may disconnect a battery pack 112 in response to a determination that the temperature of the battery pack 112 exceeds a threshold temperature. By way of another example, the system controller 302 may disconnect a battery pack 112 in response to a determination that a current being exchanged between the battery pack 112 and another battery pack 112 exceeds a threshold current.

In some embodiments, the system controller 302 disconnects one or more battery packs 112 from the loads 122 in response to the system receiving a current input from the loads 122 (e.g., a regenerative current, a braking current, a regenerative braking current, etc.). Specifically, the system controller 302 may disconnect one or more battery packs 112 to control which battery pack 112 receives and is charged by the regenerative current. By way of example, the system controller 302 may identify the battery pack 112 having the lowest voltage or state of charge and disconnect all of the other battery packs 112. In this way, the system controller 302 ensures that the battery pack 112 having the most available capacity to store energy is the recipient of the regenerative current.

A user may equip the system 100 with a depleted battery pack 112 in order to facilitate regenerative braking. By way of example, in a condition where the system 100 is equipped with only full battery packs 112 (e.g., battery packs 112 having a 1000% state of charge), when a regenerative current is provided, the battery packs 112 may not have available capacity to store additional energy. Accordingly, the energy supplied by the regenerative current may not be efficiently captured (e.g., may be lost as thermal energy). Instead, a user may replace one of the full battery packs 112 with a partially or completely depleted battery pack 112 that can receive the regenerative current. While removing the full battery pack 112 may reduce an overall state of charge of the system 100 (e.g., resulting in a lesser runtime for the system), replacing the full battery pack 112 with the depleted battery pack 112 may permit additional regenerative current functionality.

In a fourth battery discharging strategy according to an exemplary embodiment, the system controller 302 alternates between connecting two battery packs 112. By way of example, the system controller 302 may alternate between (a) connecting a first battery pack 112 for a first duration while a second battery pack is disconnected and (b) connecting the second battery pack 112 for a second duration while the first battery pack is disconnected. The lengths of the first duration and the second duration (e.g., the ratio between the first duration and the second duration) may vary based on a state of charge or voltage of each battery pack 112. By way of example, the first duration may be greater than the second duration if the state of charge of the first battery pack 112 is greater than the state of charge of the second battery pack 112. In one example, the system controller 302 discharges a high voltage battery pack 112 for 6 seconds, then subsequently discharges a lower voltage battery pack 112 for 2 seconds, then repeats this alternating process over time.

The system controller 302 may control the opening and closing of the battery loading switches 126 in order to control the discharge rates of the corresponding battery packs 112 to bring two or more battery packs 112 to the same voltage. By way of example, the battery loading switch 126a could be closed for a greater amount of time than the battery loading switch 126b, causing the state of charge of the battery pack 112a to decrease more quickly than the state of charge of the battery pack 112b. When the battery packs 112a and 112b reach the same state of charge (or within a threshold of each other), the system controller 302 may close the battery loading switches 126a and 126b to connect the battery packs 112a and 112b in parallel.

### Communication Between System Controller and Battery Controllers

According to a third set of control strategies, discussed herein as communication strategies, each of the battery packs 112 includes a battery controller 310 that communicates information (e.g., operating characteristics) of the corresponding battery pack 112 with other components of the system 100. By way of example, the battery controllers 310 may communicate information between one another and to the system controller 302. The system 100 may utilize various strategies to control communication of information from the battery controllers 310 to the system controller 302. Such strategies may seek to increase the efficiency of communication (e.g., by preventing multiple battery controllers 310 from all communicating directly with the system controller 302) and/or the robustness of the system 100 (e.g., the ability of the system to operate when a battery pack 112 is connected or disconnected from the system 100). Multiple communication strategies are described herein, and the system 100 may utilize one communication strategy or multiple communication strategies simultaneously and/or may be capable of switching between communication strategies (e.g., in response to a user selection).

In a first communication strategy according to an exemplary embodiment, one of the battery controllers 310 is designated as a primary controller or leader controller. The other battery controllers 310 may be designated as secondary controllers or follower controllers. In some embodiments, the leader controller is the battery controller 310 associated with a predetermined receptacle 114. By way of example, the battery controller 310 of any battery pack 112 that is coupled to the predetermined receptacle 114 may be assigned as the leader controller. In such an example, battery packs 112 may be added to or removed from other receptacles 114 without disrupting operation of the system 100. In other embodiments, the battery controller 310 of a specific battery pack 112 is permanently designated as the leader controller (e.g., regardless of which receptacle 114 receives the battery pack 112). In such an example, other battery packs 112 may be added to or removed from the system 100 without disrupting operation of the system 100 as long as the battery pack 112 with the leader controller remains connected.

The leader controller communicates with the other battery controllers 310 (e.g., one or more follower controllers), receiving information (e.g., operating characteristics) from the other battery controllers 310. By way of example, the battery controllers 310 may form a mesh network, such that the battery controllers 310 can communicate with one another directly or through another battery controller 310. The leader controller may compile the information from the other battery controllers 310, as well as information regarding the battery pack 112 associated with the leader controller, and send compiled information to the system controller 302. In this way, the leader controller is the only battery controller 310 that communicates directly with the system controller 302, and the other battery controllers 310 communicate indirectly with the system controller 302 through the leader controller.

In a second communication strategy according to an exemplary embodiment, the leader controller designation is dynamically assigned (e.g., changes between different battery controllers 310 in response to certain conditions). The system 100 may operate according to predetermined logic to select the leader controller according to one or more predetermined criteria. By way of example, the system 100 may select the battery controller 310 associated with the battery pack 112 having the highest level of charge. By way of another example, the system 100 may select the battery controller 310 associated with the battery pack 112 having the lowest temperature. By way of another example, the system 100 may select the battery controller 310 based on identification data (e.g., a serial number) of the associated battery pack 112. In one such example, the system 100 selects the battery controller 310 associated with the battery pack 112 having highest serial number (e.g., indicating that the battery pack 112 is newest) as the leader controller.

In some embodiments, the system 100 selects the leader controller based on a single criterion. In other embodiments, the system 100 selects the leader controller based on multiple criteria. By way of example, the system 100 may select the leader controller using criteria in stages according to a decision tree. The system 100 may evaluate based on a first criterion (e.g., charge level). If the first evaluation is inconclusive (e.g., the charge levels of two battery packs 112 are not sufficiently different from one another), the system 100 may proceed to a second evaluation based on a second criterion (e.g., battery pack temperature). If the second evaluation is inconclusive, the system 100 may proceed to a third evaluation based on a third criterion. This process may be repeated using different criteria until the leader pack is selected. By way of another example, the system 100 may calculate a score for each battery controller 310 based on a combination of multiple criteria (e.g., according to a predetermined formula). In one such embodiment, each criterion is assigned a weight, and the collective weighted criteria are used to calculate the score. The battery controller 310 with the highest score may be selected as the leader controller.

In some embodiments, the evaluation to select the leader controller is performed by all of the battery controllers 310 connected to the system 100. The battery controllers 310 may perform the evaluation periodically (e.g., 100 times per second, once per second, once per minute, etc.). The battery controllers 310 may all perform the evaluation simultaneously. Alternatively, the battery controllers 310 may perform the evaluation at staggered times (e.g., not simultaneously).

If all of the battery controllers 310 perform the same evaluation utilizing the same, shared data, the battery controllers 310 may all reach the same conclusion regarding the selection of the leader controller. Accordingly, each battery controller 310 may self-assign as a leader controller or a follower controller (e.g., without a command from another battery controller 310). As such, each battery controller 310 may independently determine which battery controller 310 should share information with the system controller 302 and when information should be shared with the system controller 302.

In the first communication strategy or the second communication strategy, the leader controller may communicate information regarding each battery pack 112 of the system 100 to the system controller 302. The leader controller may associate each piece of provided information with a specific battery pack 112. By way of example, the leader controller may provide one or more operating characteristics (e.g., battery temperature, charge level, etc.) along with an indicator identifying the battery pack 112 associated with the operating characteristics.

In other embodiments, the battery controllers 310 cooperate to calculate a virtual pack state that collectively represents the battery packs 112 of the system 112. The virtual pack state may have operating characteristics that approximate the collective operating characteristics of the battery packs 112 of the system 100. By way example, the virtual pack state may have a state of charge that represents an overall state of charge of all of the battery packs 112. By way of another example, the virtual pack state may have a battery temperature that represents an average temperature of the battery packs 112 or the highest temperature of the battery packs 112. By communicating the virtual pack state instead of the operating characteristics of each individual battery pack 112, the amount of data communicated between the battery controllers 310 and the system controller 302 may be reduced while still providing operating characteristics representative of all of the battery packs 112.

In a third communication strategy according to an exemplary embodiment, the system controller 302 communicates directly with each of the battery controllers 310. By way of example, the system controller 302 may receive the operating characteristics of the battery pack 112 directly from the corresponding the battery controller 310 (e.g., without the operating characteristics being passed through another battery controller 310). In some embodiments, the system controller 302 includes a first controller (e.g., within the battery tray 60) in communication with a second controller (e.g., within a piece of power equipment). The first controller may collect information (e.g., operating characteristics) from each of the battery controllers 310. The first controller may compile the information and provide the compiled information to the second controller.

In some embodiments, the system 300 switches between multiple communication strategies. By way of example, the system 300 may default to operating according to the first communication strategy. The system 100 may switch to the third communication strategy in response to an indication that a leader controller is not available. By way of example, the battery controllers 310 may determine that a leader controller is not present (e.g., due to the battery pack 112 containing a designated leader controller being removed). In response to such a determination, one or more of the battery controllers 310 may instruct the system controller 302 to begin communication according to the third communication strategy. Accordingly, the system 300 may benefit from the efficient communication of the first communication strategy while still being able to switch to the third communication strategy to continue operating in the event that the leader controller is removed.

In some embodiments, the system 300 varies the interval or delay between communications between the battery controllers 310 and the system controller 302. By way of example, the system 300 may increase the time delay between communications in response to the state of charge of a battery pack 112 falling below a threshold state of charge. By way of another example, the battery controller 310 (e.g., a leader controller) may only initiate communication with the system controller 302 when an operating characteristic of one of the battery packs 112 has varied greater than a threshold amount. In this way, the system controller 302 may operate under the assumption that the operating characteristics remain constant until receiving an indication to the contrary from the battery controller 310. Such a strategy may facilitate operation of the system 300 while minimizing power consumption due to communication between the system controller 302 and the battery controllers 310.

### Selection of Control Strategy Based on Power Equipment Application

In some embodiments, the system 300 selects a control strategy (e.g., changes between multiple control strategies) based on the current power equipment application of the system 300. By way of example, upon connection of a battery pack 112 to the system 300, the corresponding battery controller 310 may attempt to retrieve information regarding the power equipment application of the system 300 (e.g., from the system controller 302, from the power equipment identifier 2130, etc.). Based upon the information regarding the power equipment application, the battery controller 310 determines a control strategy for the system 300. By way of another example, a preferred control strategy for the power equipment 400 may be predetermined (e.g., based on the power equipment application associated with the power equipment 400) and stored (e.g., by the system controller 302). Upon connection of a battery pack 112 to the system 300, the corresponding battery controller 310 may receive a command from the system controller 302 instructing the battery controller 310 to operate according to the preferred control strategy.

In some embodiments, certain control strategies, referred to herein as predictable load control strategies, are preferred for use in power equipment applications having predictable loads. One example of a type of power equipment 400 having a predictable load is a light tower (e.g., the light tower 412). When operating at a given setting (e.g., a given lumen output), a light tower may require a predictable (e.g., substantially constant) amount of power. As long as the predictable load is satisfied by the system 300, there may be no detectable (e.g., by an operator) decrease in performance of the light tower due to variations in the state of charge of a battery pack 112. Accordingly, it may be desirable to select control strategies that maximize the run time of the power equipment 400 while still satisfying the predictable load. Other examples of power equipment having predictable loads may include pumps, fans, compressors, vibratory compactors, demolition hammers, pressure washers, or other power equipment.

An example of a predictable load control strategy may include the first battery discharging strategy in which each battery pack 112 is discharged individually. Specifically, each battery pack 112 may be discharged until the state of charge of the battery pack 112 is insufficient to satisfy the predictable load. By discharging each battery pack 112 individually, the overall run time of the power equipment 400 may be maximized. Other predictable load control strategies may include battery discharging strategies where battery packs 112 can be drained individually, such as the fourth battery discharging strategy.

In some embodiments, certain control strategies, referred to herein as reactive load control strategies, are preferred for use in power equipment applications having reactive loads that vary over time in response to certain events or conditions. One example of a type of power equipment 400 having a reactive load is a lawn mower (e.g., the lawn mower 410). The power required by an electric motor that drives a lawn mower blade may vary throughout operation. By way of example, the electric motor may react to the lawn mower blade contacting an obstacle, such as a rock or a patch of tall grass, by increasing the power required by the electric motor. This increased load may be accommodated by connecting multiple battery packs 112 in parallel to supply a greater current to the load. Accordingly, it may be desirable to select control strategies that permit quickly connecting multiple battery packs 112 in parallel. Other examples of power equipment having reactive loads may include saws, vehicles, excavators, lifts, or other power equipment.

Examples of reactive load control strategies may include control strategies where multiple battery packs are joined or discharged simultaneously, such as any of the battery joining strategies, the second battery discharging strategy, or the third battery discharging strategy. By way of example, a lawnmower may typically operate at a low load state (e.g., requiring 1150 A of supply current) and occasionally change to a high load state (e.g., requiring 1300 A of supply current) in response to the lawnmower encountering an obstacle. In the low load state, a single battery pack 112 may be sufficient to supply the required power. In the high load state, it may be desirable to have multiple battery packs 112 connected to the load 122 to satisfy the increased power requirement.

An example of how the third battery joining strategy may be utilized in a power equipment application that is associated with reactive loading will now be described. Initially, the load 122 may be connected to a first battery pack 112, and a second battery pack 112 may be disconnected from the load 122. The system controller 302 may automatically connect the second battery pack 112 to the load 122 in response to a determination that the energy or current required by the load 122 exceeds a threshold level (e.g., due to the lawnmower encountering an obstacle). Accordingly, the system controller 302 may react to an increased load by reducing the load on each individual battery pack 112. The system controller 302 may monitor the current flowing between the battery packs 112 (e.g., due to the battery packs 112 being at different voltages). The system controller 302 may disconnect one of the battery packs 112 from the load 122 in response to a determination that the current passing between the battery packs 112 exceeds a threshold current (e.g., 1200 A), as it may be undesirable for the battery packs 112 to be equalizing charge between one another in certain situations.

In other embodiments, the control strategy for the system 300 is otherwise selected. By way of example, the control strategy may predetermined or preselected by a user (e.g., a manufacturer of the system 300, a distributor of the system 300, an end user of the system 300, etc.). By way of example, the control strategy may be selected through the user interface 330 or a user device in communication with the system 300, such as a smartphone, tablet, laptop computer, or desktop computer. The selected control strategy may include one or more user preferences, such as a preferred battery discharge order (e.g., which battery pack 112 should be discharged first, how many battery packs 112 should be discharged simultaneously, etc.).

### Wake/sleep Status of Battery Packs

In some embodiments, the battery controllers 310 are capable of changing the corresponding battery packs 112 between an active or wake configuration and a low power, sleep configuration. The sleep configuration may disable one or more functions of the battery packs 112 to conserve battery charge. By way of example, the sleep configuration may be utilized when the power equipment 400 is not being used (e.g., the functionality of the power equipment 400 is disabled; the battery packs 112 are not being charged and/or discharged, etc.).

In some embodiments, the system 300 utilizes the user interface 330 to determine when to switch between the sleep configuration and the wake configuration. By way of example, the user interface 330 may include a switch or key that can be moved between a first position (e.g., an on position) and a second position (e.g., an off position). In response to the key being moved to the off position, the battery controllers 310 may change the battery packs 112 to the sleep configuration. In response to the key being moved to the on position, the battery controllers 310 may change the battery packs 112 to the wake configuration.

In some embodiments, the battery controllers 310 are capable of providing commands (e.g., wake signals) between one another to change the wake/sleep state of the battery packs 112. By way of example, a first battery controller 310 (e.g., a leader controller) may transmit a wake signal to each of the other battery controllers 310 to change the corresponding battery packs 112 to the wake configuration. The wake signals may be used to wake a battery pack 112 even when the key is in the off position.

In some embodiments, one of the battery controllers 310 (e.g., the leader controller) periodically (e.g., once every 100 seconds, once per minute, etc.) wakes the corresponding battery pack 112 for a period of time (e.g., 1 second, 100 seconds, 1 minute, etc.). While the battery controller 310 is in the wake configuration, the battery controller 310 may evaluate one or more criteria to determine if the other battery packs 112 should be reconfigured into the wake configuration as well. By way of example, the battery controller 310 may determine if the charger 132 is supplying electrical energy to the charging circuit 130. In response to a determination that the charger 132 is supplying the electrical energy, the battery controller 310 may provide a wake signal to the other battery controllers 310 to facilitate charging the other battery packs 112.

### Powering Load While Charging

In some embodiments, the charger 132 charges the battery packs 112. By way of example, in response to the charger 132 providing electrical energy, the system controller 302 may close the charger switch 138 and one or more of the battery charging switches 136 to couple the corresponding battery packs 112 to the charger 132. In some embodiments, the system 300 selectively permits discharging one or more of the battery packs 112 to power the load 122 while one or more of the battery packs 112 are charged by the charger 132. In some embodiments, the system 300 determines whether to permit discharging the battery packs 112 based on the position of the key of the user interface 330. While charging, the system 300 may utilize any of the battery joining strategies and/or battery discharging strategies described herein.

When the key is in the off position, the system 300 may prevent discharging the battery packs 112. By way of example, the system controller 302 may disconnect the battery packs 112 from the loads 122 by opening the load switch 128. The charger 132 may directly power the loads 122. By way of example, the system controller 302 may close the bypass switch 150 to directly couple the charger 132 to the loads 122.

When the key is in the on position, the system 300 may permit discharging the battery packs 112 (e.g., by closing the load switch 128 and one or more of the battery loading switches 126) while the charger 132 supplies electrical energy to the system 100. A first portion of the electrical energy may be used to charge one or more of the battery packs 112 while a second portion of the electrical energy may be supplied directly to the loads 122. Because the electrical energy from the charger 132 is split between the battery packs 112 and the loads 122, the battery packs 112 may charge more slowly than when the electrical energy from the charger 132 directly charges the battery packs 112. The first portion of the electrical energy may charge the battery packs 112 more quickly than the battery packs 112 are discharged by the loads 122, such that the state of charge of the battery packs 112 increases. The first portion of the electrical energy may charge the battery packs 112 more slowly than the battery packs 112 are discharged by the loads 122, such that the state of charge of the battery packs 112 decreases. Alternatively, a first subset of the battery packs 112 may be coupled to the charger 132 and decoupled from the loads 122, such that the first subset of the battery packs 112 charge. Simultaneously, a second subset of the battery packs 112 may be decoupled from the charger 132 and coupled to the loads 122, such that the second subset of the battery packs 112 discharge.

### Low Power Auxiliary Output

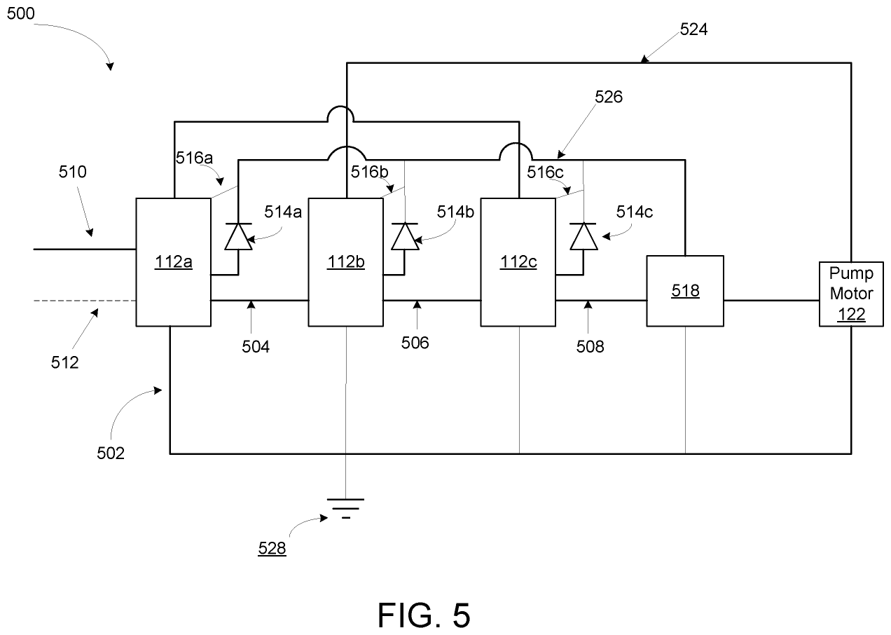

Referring now to FIG. 5, the system 500 may provide various communication systems for the battery packs 112 (and the corresponding battery controllers 310) to communicate with one another and/or with the system controller 302. FIG. 5 illustrates a portion of the system 500 including the battery packs 112, a portion of the load circuit 120, and a load 122. In some embodiments, the load 122 is shown as a pump motor (e.g., for a pressure washer or hydraulic system). It should be understood that other components of the system 100 shown in FIG. 1 (e.g., the charging circuit 130, the battery loading switches 126, etc.) may be present in the system 500 of FIG. 5, even if the components are not explicitly illustrated.

As shown in FIG. 5, the system 500 includes a CAN bus 502 that facilitates communication between the battery packs 112 (e.g., the battery controllers 310 of the battery packs 112) and/or the system controller 302. Specifically, the CAN bus 502 may facilitate digital communication between any components coupled to the CAN bus 502. As shown, the CAN bus 502 couples the battery packs 112 and the system controller 302 in series. A first section of the CAN bus 502, shown as CAN cable 504, extends between and operatively couples the battery pack 112a and the battery pack 112b. A second section of the CAN bus 502, shown as CAN cable 506, extends between and operatively couples the battery pack 112b and the battery pack 112c. A third section of the CAN bus 502, shown as CAN cable 508, extends between and operatively couples the battery pack 112c and the system controller 302. In other embodiments, the CAN bus 502 is otherwise arranged (e.g., connections are formed between different modules). In yet other embodiments, the CAN bus 502 is omitted.

As shown in FIG. 5, the system 500 further includes a first communication line or signal transmission line, shown as a low power energization line 510, and a second communication line or signal transmission line, shown as high power energization line 512. The low power energization line 510 and/or the high power energization line 512 may be operatively coupled to one or more of the battery packs 112 (e.g., to the battery controllers 310). By way of example, the low power energization line 510 and/or the high power energization line 512 may be coupled to each of the battery packs 112 individually, or to all of the battery packs 112 in series.

The low power energization line 510 may be configured to deliver a first energization signal to one or more of the battery packs 112, the first energization signal indicating that the battery pack 112 should begin delivering electrical energy to the loads 122 at a low power level (e.g., at a low current, at a low voltage, etc.). By way of example, the low power level may have a current between 500 mA and 15 A, may have a current less than 500 A, may be less than or equal to 150 W of power, etc. In some embodiments, the low power energization line 510 is operatively coupled to the system controller 302 and/or a portion of the user interface 330 (e.g., an on/off switch, a start button, a key, etc.), such that the first energization signal is initiated by the system controller 302 and/or the portion of the user interface 330. The high power energization line 512 may be configured to deliver a second energization signal to one or more of the battery packs 112, the second energization signal indicating that the battery pack 112 should begin delivering electrical energy to the loads 122 at a high power level (e.g., at a high current, at a high voltage, etc.). By way of example, the high power level may have a current between 500 A and 5000 A. In some embodiments, the high power energization line 512 is operatively coupled to the system controller 302 and/or a portion of the user interface 330, such that the system controller 302 and/or the portion of the user interface 330 initiate the second energization signal. In some embodiments, the battery packs 112 supply approximately 48V at both the high power level and the low power level.

Referring to FIG. 5, the system 500 further includes a common low power auxiliary output, sensing bus, or auxiliary line, shown as auxiliary bus 526. The auxiliary bus 526 is electrically coupled to each of the battery packs 112 and the system controller 302 in parallel. The system controller 302 may be directly coupled to the auxiliary bus 526. The battery packs 112 are electrically coupled to the auxiliary bus 526 through a series of diodes that each permit current to flow from one of the battery packs 112 to the auxiliary bus 526, but limits (e.g., prevents) current from flowing back from the auxiliary bus 526 to the battery pack 112. Specifically, the battery pack 112a is coupled to the auxiliary bus 526 through a diode 514a. The battery pack 112b is coupled to the auxiliary bus 526 through a diode 514b. The battery pack 112c is coupled to the auxiliary bus 526 through a diode 514c. The diodes 514a, 514b, and 514c may be referred to collectively herein as the diodes 514, and a generic diode may be referred to as a diode 514. In some embodiments, the auxiliary bus 526 and the diodes 514 are arranged such that the auxiliary bus 526 is coupled to the battery packs 112 regardless of whether or not the battery loading switches 126 and the battery charging switches 136 are open or closed.

In some embodiments, the power transferred from the battery packs 112 to the loads 122 has a greater magnitude (e.g., the same voltage but different currents, etc.) than the power transferred from the battery packs 112 to the auxiliary bus 526. The electrical energy delivered to the auxiliary bus 526 may be used primarily for sensing, not for driving loads, so delivering a relatively low power to the auxiliary bus 526 may facilitate the use of less robust and/or more sensitive components without negatively affecting performance of the system. In some embodiments, the battery packs 112 each deliver electrical energy to the auxiliary bus 526 at a current between 1 mA and 1 A.

Each of the battery packs 112 further includes a sensing line or voltage sensor that permits the battery pack 112 (e.g., the battery controller 310 of the battery pack 112) to sense (e.g., measure) the voltage at the auxiliary bus 526. Specifically, the battery pack 112a includes a voltage sensor 516a that measures the voltage at the auxiliary bus 526. The battery pack 112b includes a voltage sensor 516b that measures the voltage at the auxiliary bus 526. The battery pack 112c includes a voltage sensor 516c that measures the voltage at the auxiliary bus 526. The voltage sensors 516a, 516b, and 516c may be referred to collectively herein as the voltage sensors 516, and a generic voltage sensor 516 may be referred to as a voltage sensor 516. In some embodiments, voltage sensors 516 are arranged such that the voltage sensors 516 remain coupled to the auxiliary bus 526 regardless of whether or not the battery loading switches 126 and the battery charging switches 136 are open or closed.

In operation, the auxiliary bus 526 may be used to facilitate communicating information between the controllers of the system 500 without the information having to pass through a line of digital communication, such as the CAN bus 502. In some such embodiments, the battery controllers 310 utilize the auxiliary bus 526 to determine if other voltage sources (e.g., other battery packs 112, capacitors, the charger 132, other power sources or power supplies, etc.) are present within the system 500. Such as communication method may be desirable, as it may reduce traffic on the CAN bus 502 or permit omission of the CAN bus 502 entirely. In other embodiments, a different communication protocol is utilized, such as J1939, Bluetooth, CANopen, or other communication protocols.

FIG. 5 illustrates one example of the system 500 operating in this way. In this example, the battery pack 112a supplies electrical energy at 48V, the battery pack 112b supplies electrical energy at 50V, and the battery pack 112c supplies electrical energy at 58V. Due to the presence of the diodes 514, only the supplied electrical energy having the highest voltage is permitted to pass through the diodes 514 to the auxiliary bus 526. By way of example, the electrical energy from the battery pack 112c may pass through the diode 514c, as there is no greater voltage on the auxiliary bus 526. The diode 514c may introduce some resistance, such that the voltage downstream of diode 514c (e.g., 57V) is less than the voltage upstream of the diode 514c (e.g., 58V). Because the voltage on the auxiliary bus 526 (e.g., 57V) is greater than the voltage from the battery pack 112a (e.g., 48V), the diode 514a electrically decouples the battery pack 112a from the auxiliary bus 526 to prevent current from flowing backward through the diode 514a. Similarly, because the voltage on the auxiliary bus 526 (e.g., 57V) is greater than the voltage from the battery pack 112b (e.g., 50V), the diode 514b electrically decouples the battery pack 112b from the auxiliary bus 526 to prevent current from flowing backward through the diode 514b.

Each battery controller 310 may establish (e.g., calculate, predict, estimate) an a reference voltage or expected voltage range (e.g., an expected voltage with a measurement tolerance) that the corresponding battery pack 112 would provide to the auxiliary bus 526 if the battery pack 112 were the source of the highest voltage in the system 500. By way of example, the battery controller 310 may utilize an internal voltage sensor (e.g., one of the operating characteristic sensors 312) to identify an internal voltage of the battery pack 112 (e.g., which may be the voltage delivered by the battery pack 112). Based on the measured internal voltage, the battery controller 310 may determine the expected voltage range. By way of example, the expected voltage range may be the measured internal voltage minus an anticipated voltage drop across the corresponding diode 514.

Each battery controller 310 may receive the measurement of the voltage at the auxiliary bus 526 from the corresponding voltage sensor 516. The battery controller 310 may compare the voltage at the auxiliary bus 526 with the expected voltage range. If the voltage at the auxiliary bus 526 is within the expected voltage range, the battery controller 310 may determine that there are no other voltage sources delivering electrical energy to the auxiliary bus 526 at a higher voltage than the corresponding battery pack 112. This determination could mean that the corresponding battery pack 112 is the only voltage source in the system 500, or that there is another voltage source (e.g., another battery pack 112) delivering electrical energy to the auxiliary bus 526 at the same voltage as the corresponding battery pack. If the voltage at the auxiliary bus 526 is above the expected voltage range, the battery controller 310 may determine that there is another voltage source delivering electrical energy to the auxiliary bus 526 at a higher voltage than the corresponding battery pack 112. This determination means that there are multiple voltage sources in the system 500.

Returning to the example shown in FIG. 5, the battery controller 310 of the battery pack 112a may measure the internal voltage of the battery pack 112a to be 48V, and establish an expected voltage range of 47V±1V (e.g., based on predetermined characteristics of the diode 514a). Upon the voltage sensor 516a indicating that the voltage at the auxiliary bus 526 is 57V, which is greater than the expected voltage range, the battery controller 310 may determine that there is at least one additional voltage source delivering electrical energy to the auxiliary bus 526 at a higher voltage than the battery pack 112a.

In the example shown in FIG. 5, the battery controller 310 of the battery pack 112b may measure the internal voltage of the battery pack 112b to be 50V and establish an expected voltage range of 49V±1V (e.g., based on predetermined characteristics of the diode 514b). Upon the voltage sensor 516b indicating that the voltage at the auxiliary bus 526 is 57V, which is greater than the expected voltage range, the battery controller 310 may determine that there is at least one additional voltage source delivering electrical energy to the auxiliary bus 526 at a higher voltage than the battery pack 112b.

In the example shown in FIG. 5, the battery controller 310 of the battery pack 112c may measure the internal voltage of the battery pack 112b to be 58V and establish an expected voltage range of 57V±1V (e.g., based on predetermined characteristics of the diode 514c). Upon the voltage sensor 516b indicating that the voltage at the auxiliary bus 526 is 57V, which is within the expected voltage range, the battery controller 310 may determine that there are no other voltage sources delivering electrical energy to the auxiliary bus 526 at a higher voltage than the battery pack 112c. This determination could mean that the battery pack 112c is the only voltage source in the system 500, or that there is another voltage source (e.g., another battery pack 112) delivering electrical energy to the auxiliary bus 526 at approximately the same voltage as the battery pack 112c.

The system controller 302 may also measure the voltage at the auxiliary bus 526 (e.g., using one or more voltage sensors). If the voltage at the auxiliary bus 526 is equal to the voltage at the common C, the system controller 302 may determine that there are no voltage sources supplying electrical energy to the auxiliary bus 526. If the voltage at the auxiliary bus 526 is greater than the voltage at the common C, the system controller 302 may determine that there is at least one power supply supplying electrical energy to the auxiliary bus 526.

In some embodiments, the battery packs 112 include components (e.g., switching elements, voltage or current reducing circuits, etc.) controlled by the battery controller 310 that facilitate varying the voltage delivered by the battery pack 112. By way of example, a battery controller 310 may disconnect the corresponding battery pack 112 from the auxiliary bus 526. By way of another example, a battery controller 310 may reduce the voltage delivered by the corresponding battery pack 112 to the auxiliary bus 526. By way of another example, a battery controller 310 may increase the voltage delivered by the corresponding battery pack 112 to the auxiliary bus 526.

In some embodiments, the battery controller 310 varies the voltage delivered by the corresponding battery pack 112 to the auxiliary bus 526 when determining if another power source is present within the system 500. By way of example, the battery controller 310 may reduce the voltage delivered by the corresponding battery pack 112 (e.g., to 50% of the normal operating voltage, to 0V, etc.). By reducing the delivered voltage, it may be easier to determine if other power sources are present within the system 500. By way of example, if the delivered voltage were dropped to OV, the expected voltage range would also be 0V. Accordingly, if there was still a non-zero voltage at the auxiliary bus 526, the battery controller 310 could determine if there was another power source regardless of the magnitude of the non-voltage delivered by the other power source.

In some embodiments, the battery controller 310 varies the voltage delivered by the corresponding battery pack 112 to the auxiliary bus 526 to communicate information (e.g., data) to other battery packs 112 and/or the system controller 302. The battery controller 310 may vary the voltage according to a predetermined pattern that can be recognized by the other battery controllers 310 and/or the system controller 302. By way of example, the battery controller 310 may perform pulse-width modulation of the voltage delivered by the corresponding battery pack 112. Accordingly, the battery controller 310 may communicate information without relying upon the CAN bus 502.

In some embodiments, the battery controller 310 is configured to change a control strategy of the system 500 based on the determination of whether or not another power source is present within the system 500. By way of example, the battery controller 310 may switch between any of the battery joining strategies, battery discharging strategies, or communication strategies described herein. By way of another example, the battery controller 310 may modify a parameter (e.g., a setpoint, a threshold, etc.) of the control strategy.

In some embodiments, the battery controller 310 is configured to control the system 500 to utilize a first control strategy when the corresponding battery pack 112 is the only power source within the system 500. The battery controller may be configured to control the system 500 to utilize a second control strategy when the battery controller 310 determines that there is another power source supplying electrical energy to the auxiliary bus 526. In some embodiments, the battery controller 310 changes control strategies in response to a determination that another power source has been added to the system 500. In some embodiments, the battery controller 310 changes control strategies in response to a determination that a power source has been removed from the system 500.

### Battery Charging and Discharging Methods

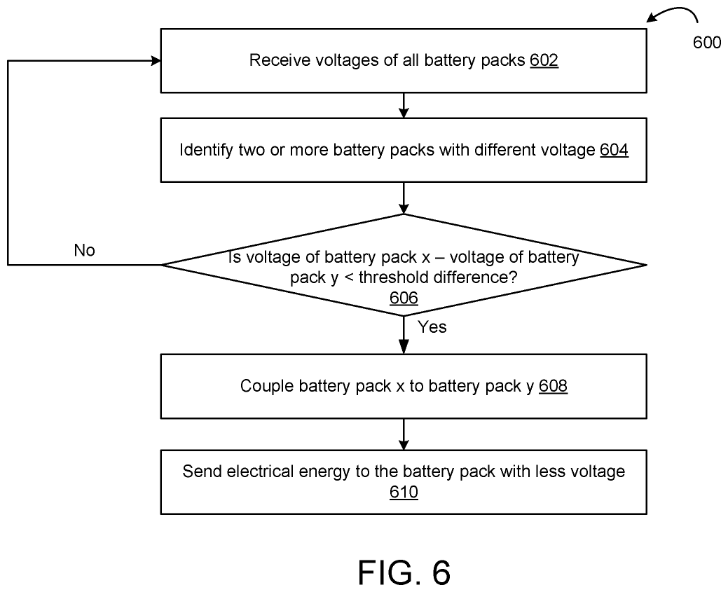

Referring now to FIGS. 6-12, flowcharts illustrating several battery charging and discharging strategies which can be used by the system controller 302 and/or the battery controller 310 are shown, according to various exemplary embodiments. The battery charging and discharging strategies may utilize one or more of the battery packs 112 and may include charging or discharging multiple battery packs 112 in parallel and/or in series. Several of the battery charging and discharging strategies illustrated in FIGS. 6-12 are referred to as “joining” strategies because they involve multiple battery packs 112 (e.g., joining multiple battery packs 112 together and/or disconnecting multiple battery packs 112 from one another). However, it should be understood that any of the strategies described with reference to FIGS. 6-12 may utilize one or more of the battery packs 112 and are not limited to embodiments that require multiple battery packs 112.

Referring now to FIG. 6, a method 600 for charging or joining batteries is shown, according to an exemplary embodiment. The system controller 302 and the battery controller 310 may implement the method 600, at least in part. The system controller 302, in part, may facilitate the opening and closing of the battery loading switch 126 and the battery-charging switch 136. Furthermore, the system controller 302 may connect and disconnect the battery packs 112 to the load bus 124 and the charging bus 134. The battery controller 310 in part, may facilitate the monitoring and reporting of the operating characteristics received by the operating characteristic sensors 312.

The method 600 begins at step 602 with the system controller 302 receiving the voltages of all the battery packs 112a, 112b, 112c. The battery controller 310 may send the voltage from the battery pack 112 through the communications interface 318 to the system controller 302. Each battery pack 112 may have different voltages from the others, and the system controller 302 may manage each voltage received separately. The system controller 302 may display the voltage measurements in a user interface (e.g., user interface 330) wherein the voltage measurements may be included within the information from an operating characteristic sensor 312.

At step 604, the system controller 302 may compare each voltage received and identify if two or more battery packs have different voltage. The system controller 302 receives a threshold voltage from memory 306. For ease of description, a threshold voltage, may be a predetermined value or calculated in real-time by the battery controller 310. The threshold voltage may be measured from the respective operating limits, which define acceptable operating ranges or limits for current, voltage, and/or power of the battery pack 112. The threshold voltage may be calculated based a state of charge, a temperature, and a load or usage time of the battery pack 112.

At step 606, the system controller 302 may calculate the voltage difference between the identified battery packs 112. The voltage difference is compared to the threshold voltage described herein. If the voltage difference between the battery packs 112 is greater than (or equal to) the threshold difference stored in memory 306, the method 600 will return to step 602. In some embodiments, a difference voltage greater than the threshold voltage indicates the battery packs 112 do not need to be joined for charging or discharging. If the voltage difference is less than the threshold voltage stored in memory 306, the method 600 will proceed to step 608. At step 608, the system controller 302 will couple the battery packs 112, which have different voltages lower than the threshold voltage stored in memory 306. The battery packs 112 may be coupled through the load bus 124 and/or through the charger bus 134.

At step 610, the system controller 302 may send electrical energy to the battery pack 112a, 112b, or 112c with the lowest voltage in the battery packs 112, identified in step 604. The electrical energy may arrive from one or more battery packs in the group of identified battery packs with the lowest voltage. The system controller 302 may keep the battery-charging switch 136 open/closed for a set duration or calculate the duration in real time using the operating characteristics, which receive data from the operating characteristic sensors 312. The battery pack will charge until the voltages of all the battery packs equalize. By way of example, the system controller 302 may identify one battery pack 112 at 82V and another battery pack 112 at 70V. By way of example, the system controller 302 may identify two battery packs 112 at 80V, and another battery pack 112 at 75V. In response to such a determination, the system controller 302 may control the switching elements to electrically couple the identified battery packs 112. Accordingly, the system controller 302 may electrically couple two battery packs 112 in response to a determination that a difference between the voltages of the two battery packs 112 is less than a threshold difference (e.g., 0V, 1V, 2V, 100V, etc.). One or more battery packs 112 having a relatively higher voltage may be electrical energy coupled to one or more battery packs 112 having a relatively lower voltage (e.g., through the load bus 124 and/or the charger bus 134) until the voltages of all of the battery packs 112 equalize (e.g., become equal). This strategy may be utilized in a situation where it would be advantageous to have multiple battery packs 112 at the same voltage.

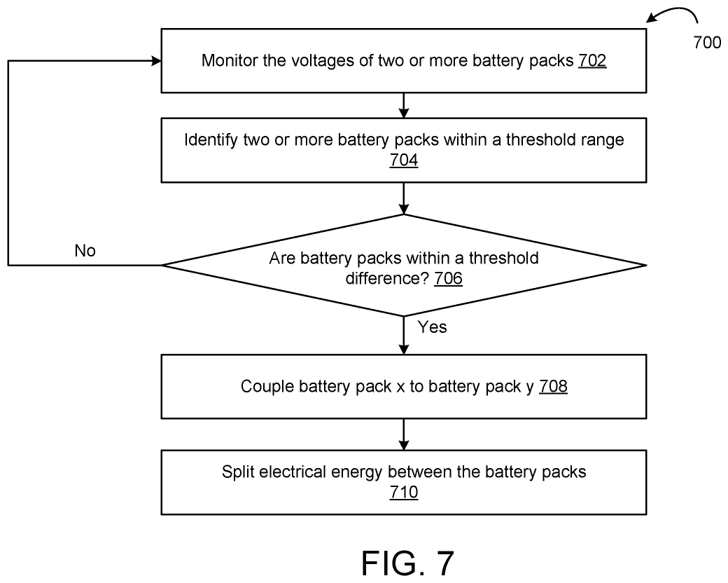

Referring now to FIG. 7, another method 700 for charging or joining batteries is shown, according to an exemplary embodiment. The system controller 302 and the battery controller 310 may implement the method 700, at least in part. The system controller 302, in part, may facilitate the opening and closing of the battery loading switch 126 and the battery-charging switch 136. Furthermore, the system controller 302 may connect and disconnect the battery packs 112 to the load bus 124 and the charging bus 134. The battery controller 310 in part, may facilitate the monitoring and reporting of the operating characteristics received by the operating characteristic sensors 312.

The method 700 begins at step 702 with the system controller 302 monitoring the voltages of all the battery packs 112a, 112b, 112c. The battery controller 310 may send the voltage from the battery packs 112 through the communications interface 318. The system controller 302 may display the voltage measurements in a user interface (e.g., user interface 330) wherein the voltage measurements may be included within the information from an operating characteristic sensor 312.

At step 704, the system controller 302 may compare each voltage received in step 702 and identify if two or more battery packs 112 are within a threshold voltage range. The system controller 302 receives a threshold voltage range from memory 306. For ease of description, a threshold voltage range, may be a predetermined range of values or calculated in real-time by the battery controller 310. A threshold voltage range may be measured from the respective operating characteristics, which define real time operating ranges or limits for current, voltage, and/or power of the battery pack 112, received from the operating characteristics sensors 330. The threshold voltage range may be calculated based a state of charge, a temperature, and a load or usage time of the battery pack 112.

At step 706, the battery controller 312 may calculate the voltage difference between the identified battery packs 112. The voltage difference is compared to the threshold voltage range described herein. If the voltage difference between the battery packs 112 are not within the threshold voltage range, defined in memory 306, the system 700 will return to step 702. In some embodiments, voltages not within the threshold voltage range indicates the battery packs 112 may not join for charging or discharging. If the voltage difference is less than the threshold voltage range defined in memory 306, the system 700 will proceed to step 708.

At step 708, the system controller 302 will couple the battery packs 112, which have voltages within the threshold voltage range stored in memory 306. The battery packs 112 may be coupled through the load bus 124 and/or through the charger bus 134. Moreover, couple battery packs 112 may charge or discharge electrical energy dependent upon the respective battery loading switch 136 and battery charging switch 126.

At step 710, the system controller 302 may split the electrical energy between the two coupled battery packs 112 identified in step 704. In some embodiments, the electrical energy may flow from a battery pack 112 with a high voltage to a battery pack 112 with a low voltage. The battery controller 310 may monitor the performance of the battery packs 112 to ensure the battery packs are operating within normal limits, defined in memory 306. In response to detecting that the voltages of two battery packs 112 are within a threshold range of one another (e.g., within 100V of one another, within 5V of one another, within 1V of one another, substantially the same voltage, etc.), the system controller 302 controls the switching elements to electrically couple the identified battery packs 112 to one another. Accordingly, the system controller 302 may electrically couple two battery packs 112 in response to a determination that a difference between the voltages of the two battery packs 112 is less than a threshold difference. This strategy may facilitate maintaining equal voltages across multiple battery packs 112 over time. Once two or more battery packs 112 have equal voltages, the rate at which the voltages change may be reduced because a supply of electrical energy from the charger 132 or a drain of electrical energy from the loads 122 will be split across multiple battery packs 112.

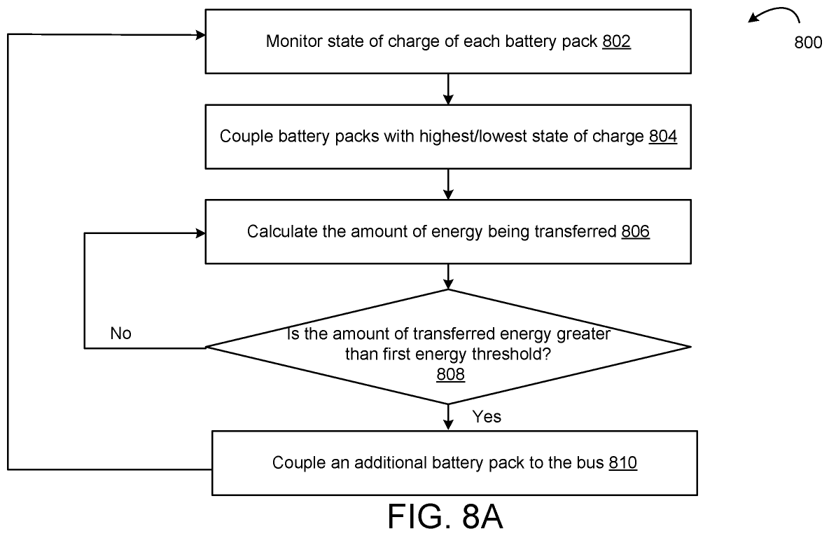

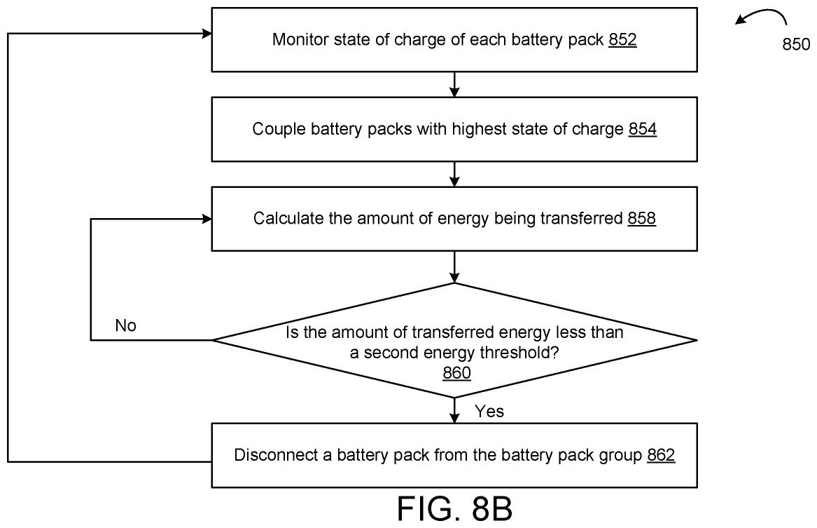

Referring now to FIG. 8A, another method 800 for charging or joining batteries is shown, according to an exemplary embodiment. The system controller 302 and the battery controller 310 may implement the method 800, at least in part. The system controller 302, in part, may facilitate the opening and closing of the battery loading switch 126 and the battery-charging switch 136. Furthermore, the system controller 302 may connect and disconnect the battery packs 112 to the load bus 124 and the charging bus 134. The battery controller 310 in part, may facilitate the monitoring and reporting of the operating characteristics received by the operating characteristic sensors 312.

The method 800 begins at step 802 with the system controller 302 receiving the state of charge all the battery packs 112a, 112b, 112c. The battery controller 310 may send the state of charge from the battery packs 112 through the communications interface 318 to the system controller 302. The system controller 302 may display the state of charge measurements in a user interface (e.g., user interface 330) wherein the state of charge measurements may be included within the information from an operating characteristic sensor 312.

At step 804, the system controller 302 will couple the battery packs with the highest/lowest state of charge. The battery packs 112 may be coupled through the load bus 124 and/or through the charger bus 134. The coupling of two or more battery packs 112 create a first subset of battery packs 112. The coupled battery packs begin transferring electrical energy to and from each other at a defined transfer rate in memory 306, through the load bus 124 and/or the charger bus 134. The battery controller 310 may increase or decrease the transfer rate dependent upon operating characteristics received from the operating characteristic sensors 312.

At step 806, the battery controller 310 may use information taken from the operating characteristic sensors 312 to calculate the amount of electrical energy transferred between the battery packs 112 within the first subset, the battery pack group, or the remaining battery packs not in a battery pack group. The rate at which the electrical energy travels may differ in certain real time conditions. For example, when multiple battery packs 112 are coupled to allow one battery pack to charge, the rate of transfer may increase. Furthermore, the rate of energy transfer may reduce when two battery packs are coupled and have the same voltage. The battery controller 310 may regulate the rate of electrical energy transferring through the battery packs 112.

At step 808, the system controller 302 may compare the amount of electrical energy transferred between the battery packs 112 within the battery pack group to an energy threshold defined in memory 306. In some embodiments, the memory 306 may contain operating limits for electrical energy transferred between battery packs 112 in the battery pack group. If the rate of electrical energy transfer in the battery group is less than (or equal to) the first energy threshold, the method 800 will return to step 806. The method 800 may repeat these two steps until the rate exceeds the first energy threshold. If the rate of electrical energy transfer in the battery group is greater than the first energy threshold, the method 800 will proceed to step 810.

At step 810, an additional battery pack 112 is coupled to the first subset of battery packs 112. For ease of description, the additional battery pack 112 coupled to the first subset of battery packs is referred to as a battery pack group (i.e., a group that includes both the first subset of battery packs 112 and the additional battery pack 112). The additional battery pack 112 may be coupled through the load bus 124 and/or through the charger bus 134. In some embodiments, the additional battery pack may have the highest/lowest state of charge. The battery pack group may transfer electrical energy to and from each other through the load bus 124 and the charger bus 134. The battery controller 310 may monitor the performance of the coupled battery packs 112 may display any warning messages regarding state of charge, temperature, load/usage time, or voltage to the user interface 330. Furthermore, the battery controller 310 may calculate the rate in which energy is transferred throughout the battery packs 112 regardless of whether the battery packs 112 are coupled or not.

At step 812, the system controller 302 will adjust the rate of energy transfer within the battery pack group to stay below the current limit, defined in the first energy threshold. For ease of description, the first energy threshold may define a current, a voltage, or a power limit, which cannot be exceeded. The battery controller 310, will monitor the operation characteristics to aid the system controller 302 to determine whether the rate of electrical energy transfer will be reduced or prevented. The method 800 will return to step 802 to monitor the charge of each battery pack 112 in order to create a new first subset. The method 800 will execute continuously over time.

Referring now to FIG. 8B, another method 850 for charging or joining batteries is shown, according to an exemplary embodiment. The system controller 302 and the battery controller 310 may implement the method 850, at least in part. The system controller 302, in part, may facilitate the opening and closing of the battery loading switch 126 and the battery-charging switch 136. Furthermore, the system controller 302 may connect and disconnect the battery packs 112 to the load bus 124 and the charging bus 134. The battery controller 310 in part, may facilitate the monitoring and reporting of the operating characteristics received by the operating characteristic sensors 312.

The method 850 begins at step 852 with the system controller 302 receiving the state of charge all the battery packs 112a, 112b, 112c. The battery controller 310 may send the state of charge from the battery packs 112 through the communications interface 318. The system controller 302 may display the state of charge measurements in a user interface (e.g., user interface 330) wherein the voltage measurements may be included within the information from an operating characteristic sensor 312.

At step 854, the system controller 302 will couple the battery packs with the highest state of charge. The battery packs 112 may be coupled through the load bus 124 and/or through the charger bus 134. The coupling of two or more battery packs 112 create a first subset of battery packs 112. The coupled battery packs begin transferring electrical energy to and from each other at a defined transfer rate in memory 306, through the load bus 124 and/or the charger bus 134.

At step 856, an additional battery pack 112 is coupled to the first subset of battery packs 112. For ease of description, the additional battery pack 112 coupled to the first subset of battery packs, known as battery pack group. The additional battery pack 112 may be couple through the load bus 124 and/or through the charger bus 134. In some embodiments, the additional battery pack may have the highest state of charge. The battery pack group may transfer electrical energy to and from each other through the load bus 124 and the charger bus 134. The battery controller 310 monitor the performance of the couple battery packs 112 may display any warning messages regarding state of charge, temperature, load/usage time, or voltage to the user interface 330. Furthermore, the operating characteristic sensors 312 may calculate the rate in which energy is transferred throughout the battery packs 112 regardless of whether the battery packs 112 are coupled or not.

At step 858, the battery controller 310 may use information taken from the operating characteristic sensors 312 to calculate the amount of electrical energy transferred between the battery packs 112 within the first subset, the battery pack group, or the remaining battery packs not in a battery pack group. The rate at which the electrical energy travels may differ in certain real time conditions. For example, when multiple battery packs 112 are coupled to allow one battery pack to charge, the rate of transfer may increase. Furthermore, the rate of energy transfer may reduce when two battery packs are coupled and have the same voltage. The system controller 302 will regulate the rate of electrical energy transferring through the battery packs 112.

At step 860, the system controller 302 may compare the amount of electrical energy transferred between the battery packs 112 within the battery pack group, to a second energy threshold defined in memory 306. For ease of description, a second energy threshold may be defined in memory 306 to indicate a battery pack will be disconnected. In some embodiments, the memory 306 may contain operating limits for transferred between battery packs 112 in the battery pack group. If the rate of electrical energy transfer in the battery group is greater than (or equal to) the second energy threshold, the method 850 will again execute step 858. The method 800 may repeat these two steps until the rate reduced below the second energy threshold. If the rate of electrical energy transfer in the battery group is less than the second energy threshold, the method 850 will proceed to step 862.

At step 862, the system controller 302 may disconnect a battery pack 112 from the battery pack group. For ease of description, a disconnected battery does not need to have energy transferred to or from the other battery packs 112 inside of the battery pack group. The disconnected battery may be the battery pack 112 added during step 856, wherein the remaining battery packs 112 coupled would be in the first subset.

By example, the system controller 302 initially connects a first subset of the battery packs 112 (e.g., one battery pack 112, multiple battery packs 112) to one of the buses (e.g., the load bus 124, the charger bus 134). The first subset may include fewer than the total number of battery packs 112 available in the system 100. The system controller 302 may select the battery packs 112 of the first subset based on various predetermined criteria. By way of example, the system controller 302 may select one or more battery packs 112 having the highest state of charge for use in the first subset. By way of another example, the system controller 302 may select one or more battery packs 112 having the lowest state of charge for use in the first subset.

Referring again to FIG. 8A, in response to a first predetermined condition (e.g., the amount of energy transfer exceeds a threshold in step #808, the system controller 302 couples an additional battery pack 112 to the bus. The combination of the first subset and the additional battery pack 112 may be referred to as a battery pack group. By utilizing the battery group instead of the first subset, the total current transfer through each battery pack 112 may be reduced. Additionally, battery packs 112 outside of the first subset may be charged or discharged. Conversely, in the method 850 of FIG. 8B, in response to a second predetermined condition (e.g., the amount of energy transfer is less than a second threshold in step 860), the system controller 302 may disconnect one or more of the battery packs 112. The system controller 302 may disconnect the additional battery pack 112, returning the system 100 to utilizing the first subset. Alternatively, the system controller 302 may disconnect a battery pack of the first subset, causing the system 100 to utilize a second subset different from the first subset.

In some embodiments, the first predetermined condition relates to an amount of energy being transferred through the bus (e.g., energy transferred per second, power, watts, etc.). The system controller 302 continuously monitors the amount of energy being transferred through the bus (e.g., from the battery packs 112 of the first subset, into the battery packs of the first subset). By way of example, the system controller 302 may calculate the amount of energy being transferred based on voltage and current measurements within each battery pack 112 from the operating characteristic sensors 312. The system controller 302 may compare the amount of energy being transferred to a first threshold amount of energy (e.g., an upper bound on the desired energy transfer range for the number of batteries that are currently coupled to the bus). In response to the amount of energy being transferred exceeding the first threshold amount of energy, the system controller 302 may determine that the first predetermined condition has been met. The first threshold amount of energy may be based upon a charge current limit and/or a discharge current limit of one or more of the battery packs 112 (e.g., stored in the memory 316). By changing from the first subset to the battery pack group, the system 100 may prevent the energy transfer from causing the battery packs 112 to exceed the charge current limit or the discharge current limit (e.g., thereby performing load sharing or peak shaving).

In some embodiments, the second predetermined condition relates to the amount of electrical energy transferred through the bus. The system controller 302 may compare the amount of electrical energy transferred to a second threshold amount of energy (e.g., a lower bound on the desired energy transfer range for the number of batteries that are currently coupled to the bus). In response to the amount of energy being transferred falling below the second threshold amount of energy, the system controller 302 may determine that the second predetermined condition has been met. The second threshold amount of energy may be lower than the first threshold amount of energy. Alternatively, the second threshold amount of energy may be the same as the first threshold amount of energy. By disconnecting a battery pack 112 from the battery pack group, the state of charge of the disconnected battery pack 112 may again change independent from the charging or discharging of the remaining connected battery packs 112.

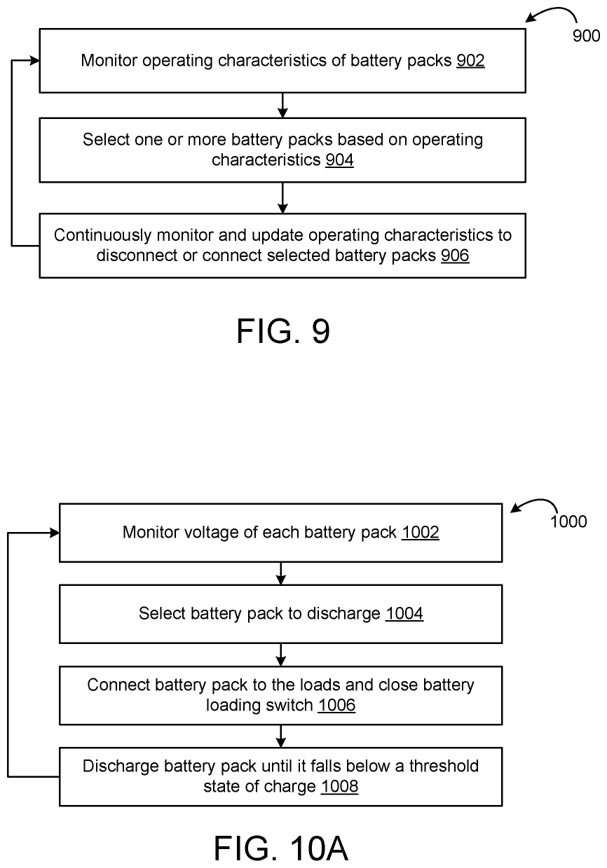

Referring to FIG. 9, another method 900 for joining or charging batteries is shown, according to an exemplary embodiment. The system controller 302 and the battery controller 310 may implement the method 900, at least in part. The system controller 302, in part, may facilitate the opening and closing of the battery loading switch 126 and the battery-charging switch 136. Furthermore, the system controller 302 may connect and disconnect the battery packs 112 to the load bus 124 and the charging bus 134. The battery controller 310 in part, may facilitate the monitoring and reporting of the operating characteristics received by the operating characteristic sensors 312.

The method 900 begins at step 902 with the system controller 302 monitors the operation characteristics all the battery packs 112a, 112b, 112c. The battery controller 310 may send the operation characteristics from the battery pack 112 through the communications interface 318, to the system controller 302. The system controller 302 may display the state of charge measurements in a user interface (e.g., user interface 330) wherein the state of charge measurements may be included within the information from an operating characteristic sensor 312.

At step 904, the system controller 302 may select one or more battery packs 112 to couple based on the operation characteristics received from the operation characteristic sensors 312. In some embodiments, the operation characteristics may not contain the voltage or state of charge. Each operation characteristic is measured to ensure the optimal battery packs 112 are connected to each other. The system controller 302 may display the voltage measurements in a user interface (e.g., user interface 330) wherein the voltage measurements may be included within the information from an operating characteristic sensor 312.

At step 906, the battery controller 310 may continuously monitor the operation characteristics sensors 312 and update the operating characteristics in real time. Furthermore, the system controller 302 may decide which battery packs to connect and/or disconnect to ensure the maximum life of each battery pack 112. The method 900 proceeds again to step 902, to allow for a continuous connection and/or disconnection of the battery packs 112. Furthermore, the method 900 may use any methods described herein dependent on the various use cases described herein.

Moreover, the system controller 302 selects one or more battery packs connects and disconnects based on operating characteristics of the battery packs 112 other than voltage/state of charge. In some embodiments, the system controller 302 connects or disconnects battery packs 112 based on the impedance of the battery pack 112. By way of example, the system controller 302 may disconnect the battery pack having the highest or lowest impedance. In some embodiments, the system controller 302 connects or disconnects battery packs 112 based on a predetermined operating characteristic of the battery pack 112, such as the composition of the battery cells in the battery pack 112 (e.g., lead-acid, Lithium-ion, etc.). By way of example, the system controller 302 may be configured to drain battery packs 112 having a first composition before draining battery packs 112 having a second composition. In some embodiments, the system controller 302 connects or disconnects battery packs 112 based on the amount of energy (e.g., an amount of energy transferred per second, power, etc.) being transferred through the battery pack 112 (e.g., a magnitude of the power flowing into or out of the battery pack 112). By way of example, the system controller 302 may disconnect a battery pack 112 in response to the amount of energy being transferred through the battery pack 112 falling below a predetermined threshold.

In some embodiments, multiple battery joining strategies may be employed in combination. By way of example, the system controller 302 may combine the third battery joining strategy and the fourth battery joining strategy. The system controller 302 may select battery packs 112 having a particular composition (e.g., lead-acid, Lithium-ion, etc.) for use in the first subset of the third battery joining strategy. In such an example, the battery packs 112 that are not included in the first subset may have a different battery composition than the battery packs 112 that are included in the first subset.

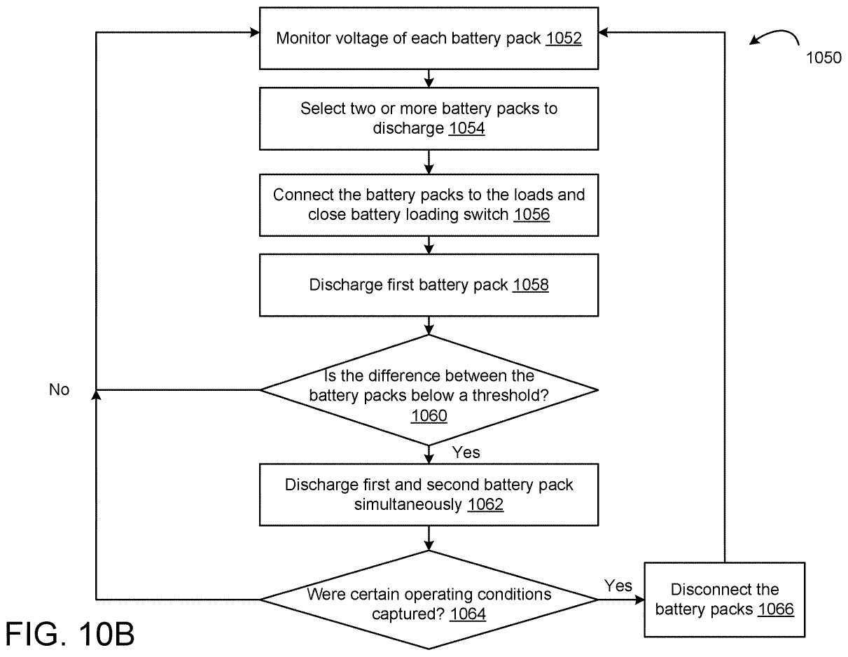

Referring to FIG. 10A, a method 1000 for discharging batteries is shown, according to an exemplary embodiment. The system controller 302 and the battery controller 310 may implement the method 1000, at least in part. The system controller 302, in part, may facilitate the opening and closing of the battery loading switch 126 and the battery-charging switch 136. Furthermore, the system controller 302 may connect and disconnect the battery packs 112 to the load bus 124 and the charging bus 134. The battery controller 310 in part, may facilitate the monitoring and reporting of the operating characteristics received by the operating characteristic sensors 312.

The method 1000 begins at step 1002 with the system controller 302 monitors the voltage of each of the battery packs 112a, 112b, 112c. The battery controller 310 may send the operation characteristics from the battery pack 112 through the communications interface 318, to the system controller 302. The system controller 302 may display the voltage measurements in a user interface (e.g., user interface 330) wherein the voltage measurements may be included within the information from an operating characteristic sensor 312.

At step 1004, the system controller 302 may select a battery pack 112 to discharge. For ease of description, the system controller 302 connecting the selected battery pack 112 to the load bus 124 executes discharging a battery pack 112. The system controller 302 may not use predetermined conditions defined in memory 306, in order to select the battery pack 112 to discharge. The system controller 302 may discharge the battery pack 112 until the state of charge is empty, or to a specified value. The battery controller 310 may use the operating characteristics of the selected battery pack 112, to calculate the duration and rate at which the battery pack 112 will discharge.

At step 1006, the system controller 302 may connect the selected battery to the loads 122 and the load bus 124. The load bus 124 may drain the battery pack 112 for a duration defined by the battery controller 310. The system controller 302 may close the battery loading switch 126 to connect the battery pack 112 to the load bus 124.

At step 1008, the selected battery pack 112, may discharge the battery until it falls below a threshold state of charge. The threshold state of charge may be defined in memory 316 as a predetermined value, which updates in real time. The state of charge may update as the operating characteristic sensors 312 provide the battery controller 310 with updated measurements in real time. The operating characteristics may define the rate, which the battery pack 112 discharges. By example, if battery pack 112a is selected to discharge and it has a low temperature, then battery pack 112a may discharge at a higher rate without compromising the integrity of the battery pack 112a.

Furthermore, by way of example, the system controller 302 may select a first battery pack 112 and close the corresponding battery loading switch 126 to couple the first battery pack 112 to the loads 122. The first battery pack 112 may be discharged to power the loads 122 until the first battery pack falls below a threshold state of charge or voltage. In response to such a determination, the system controller 302 may disconnect the first battery pack 112 and connect a second battery pack 112 to the loads 122. This process may be repeated for each battery pack 112.

Referring to FIG. 10B, another method 1050 for discharging batteries is shown, according to an exemplary embodiment. The system controller 302 and the battery controller 310 may implement the method 1050, at least in part. The system controller 302, in part, may facilitate the opening and closing of the battery loading switch 126 and the battery-charging switch 136. Furthermore, the system controller 302 may connect and disconnect the battery packs 112 to the load bus 124 and the charging bus 134. The battery controller 310 in part, may facilitate the monitoring and reporting of the operating characteristics received by the operating characteristic sensors 312.

The method 1050 begins at step 1052 with the system controller 302 monitors the voltage of each of the battery packs 112a, 112b, 112c. The battery controller 310 may send the voltage from the battery pack 112 through the communications interface 318, to the system controller 302. The system controller 302 may display the voltage measurements in a user interface (e.g., user interface 330) wherein the voltage measurements may be included within the information from an operating characteristic sensor 312.

At step 1054, the system controller 302 may select two or more battery packs 112 to discharge. For ease of description, the system controller 302 connects the selected battery packs 112 to the load bus 124 and discharges the battery packs 112. The system controller 302 may not use predetermined conditions defined in memory 306, in order to select the battery pack 112 to discharge. The system controller 302 may discharge the battery packs 112 until the state of charge is empty, or to a specified value. The battery controller 310 may use the operating characteristics of the selected battery packs 112, to calculate the duration and rate at which the battery packs 112 will discharge.

At step 1056, the system controller 302 may connect the selected battery packs 112 to the loads 122 and the load bus 124. The load bus 124 will drain the battery packs 112 for a duration defined by the battery controller 310. The system controller 302 may close the battery loading switch 126 to connect the battery pack 112 to the load bus 124.

At step 1058, the system controller may discharge the first battery pack 112 until it falls below a threshold state of charge. The threshold state of charge may be defined in memory 306 as a predetermined value, which updates in real time. The state of charge may update as the operating characteristic sensors 312 provide the battery controller 310 with updated measurements in real time. The operating characteristics may define the rate, which the first battery pack 112 discharges. By example, if battery pack 112a is first to discharge and it has a low temperature, then the battery pack 112a may discharge at a higher rate without compromising the integrity of the battery pack 112a.

At step 1060, the system controller 302 may calculate the voltage difference between the identified battery packs 112. The voltage difference is compared to the threshold voltage described herein. If the voltage difference between the battery packs 112 is greater than (or equal to) the threshold difference stored in memory 306, the method 1050 will proceed to step 1052. In some embodiments, a difference voltage lower than threshold voltage indicates the second battery pack 112 does not need to discharge. If the voltage difference is less than the threshold voltage stored in memory 306, the system 1050 will proceed to step 1062.

At step 1062, the system controller 302 will discharge both battery packs 112 simultaneously, which have different voltages lower than the threshold voltage stored in memory 306. The battery packs 112 may be coupled through the load bus 124 to the load 122. The battery packs 112 may discharge at different rates in accordance with each battery packs 112 respective operating characteristics.

At step 1064, the battery controller 310 may analyze the measurements from the operating characteristic sensors 312. The operating characteristics may include, but are not limited to the amount of impedance, temperature, load/usage time, and life of battery. For ease of description, the life of the battery is not the state of charge but the condition of the battery after elongated use. The battery controller 310 may observe the battery packs 112 to detect when certain operating characteristics exceed predetermined limits. The limits of the operating characteristics may be defined in memory 306. If certain conditions are not captured, the method 1050 will proceed again to step 1052. This is an indication that the two battery packs 112 have discharged with no irregularities occurring during the process. If certain operating characteristics are captured, the system controller 302 will disconnect the battery packs 112, by opening the battery charge switch 136 and the battery loading switch 126 to prevent issue with the long term use of the battery. If the certain conditions are captured, then the method will proceed to step 1066. At step 1066, the system controller will disconnect both battery packs 112. The opening of the battery loading switch 126 or a disconnection to the load bus 124 may disconnect the battery packs 112.

Moreover, the system controller 302 attempts to discharge multiple battery packs 112 simultaneously. By way of example, the system controller 302 may monitor the voltages of each battery pack 112. The system controller 302 may initially discharge a first battery pack 112 (e.g., the battery pack 112 having the highest voltage). When the difference between a first voltage of the first battery pack 112 and a second voltage of a second battery pack 112 falls below a threshold difference (e.g., the first voltage and the second voltage are within a threshold range of one another), the system controller 302 couples the second battery pack 112 to the loads 122. The system controller 302 may then discharge both of the battery packs 112 together. If the system 100 includes additional battery packs 112, the additional battery packs 112 may be connected to the loads 122 when the voltage of the additional battery packs 112 falls within the threshold range of the voltage of the connected battery packs 112.

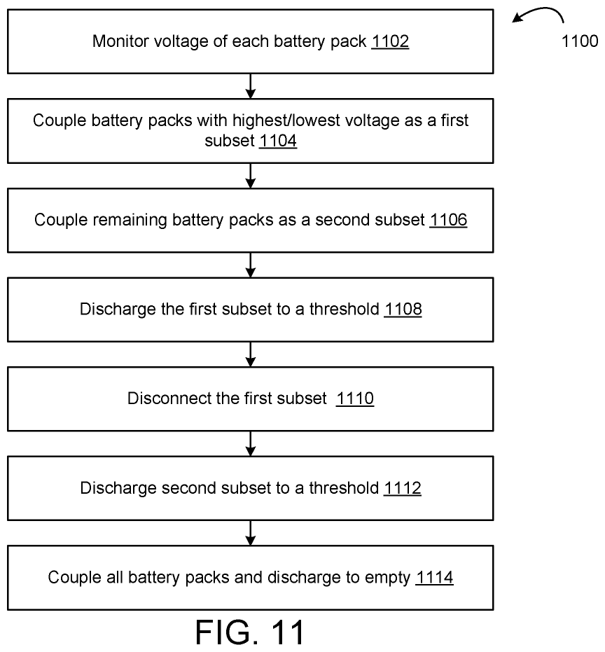

Referring to FIG. 11, another method 1100 for discharging batteries is shown, according to an exemplary embodiment. The system controller 302 and the battery controller 310 may implement the method 1100, at least in part. The system controller 302, in part, may facilitate the opening and closing of the battery loading switch 126 and the battery-charging switch 136. Furthermore, the system controller 302 may connect and disconnect the battery packs 112 to the load bus 124 and the charging bus 134. The battery controller 310 in part, may facilitate the monitoring and reporting of the operating characteristics received by the operating characteristic sensors 312.

The method 1100 begins at step 1102 with the system controller 302 monitors the voltage of each of the battery packs 112a, 112b, 112c. The battery controller 310 may send the operation characteristics from the battery pack 112 through the communications interface 318, to the system controller 302. The system controller 302 may display the voltage measurements in a user interface (e.g., user interface 330) wherein the voltage measurements may be included within the information from an operating characteristic sensor 312.

At step 1104, the system controller 302 may couple the battery packs with the highest or lowest voltage. For ease of description, the system controller 302 may couple the battery pack 112 with the highest voltage to the battery pack 112 with the next highest voltage. For ease of description, the system controller 302 may couple the battery pack 112 with the lowest voltage to the battery pack 112 with the next lowest voltage The battery packs 112 may be coupled through the load bus 124 and/or through the charger bus 134. The coupling of two or more battery packs 112 create a first subset of battery packs 112. The coupled battery packs begin transferring electrical energy to and from each other at a defined transfer rate in memory 306, through the load bus 124 and/or the charger bus 134. Similarly, at step 1106, the remaining battery packs are coupled as a second subset using the same method described herein.

At step 1108, the system controller 302, may discharge the first subset until it falls to a threshold voltage. The threshold voltage may be defined in memory 306 as a predetermined value, which updates in real time. The voltage may update as the operating characteristic sensors 312 provide the battery controller 310 with updated occurrences in real time. The operating characteristics may define the rate, which the battery pack 112 discharges. By example, if the first subset is selected to discharge and each battery pack 112 has a low temperature, then the first subset may discharge at a higher rate without compromising the integrity of the battery packs 112 within the first subset.

At step 1110, the system controller 302 will decouple each battery pack within the first subset. Each battery pack 112 may have the same voltage when decoupled as the battery packs 112 were discharged at a rate, specified by the battery controller 310, to ensure each battery pack reaches the same voltage. The battery charging switch 136 and the battery loading switch 126 may be open or closed depending on the use of each battery pack 112. Moreover, the battery packs 112 in the first subset are not connected to any battery packs 112 in the second subset.

At step 1112, the system controller 302, may discharge the second subset until it falls below a threshold voltage. The threshold voltage may be defined in memory 306 as a predetermined value, which updates in real time. The voltage may update as the operating characteristic sensors 312 provide the battery controller 310 with updated occurrences in real time. The operating characteristics may define the rate at which the battery pack 112 discharges. By example, if the second subset is selected to discharge and each battery pack has a low temperature, then the second subset may discharge at a higher rate without compromising the integrity of the battery packs 112 within the second subset.

At step 1114, the system controller 302 will couple all battery packs 112. The second subset will not be disconnected but will be couple to the battery packs 112 of the original first subset. Each battery pack may have the same voltage depending upon the requirements of the system. The system controller 302 may connect each battery to the loads 122 and open the battery charging switches 136. The battery controller 310 in each battery pack 112 determines the rate of electrical energy transfer to the loads 122. A calculation is made using the operating characteristics. Each battery pack 112 will be discharged until empty.

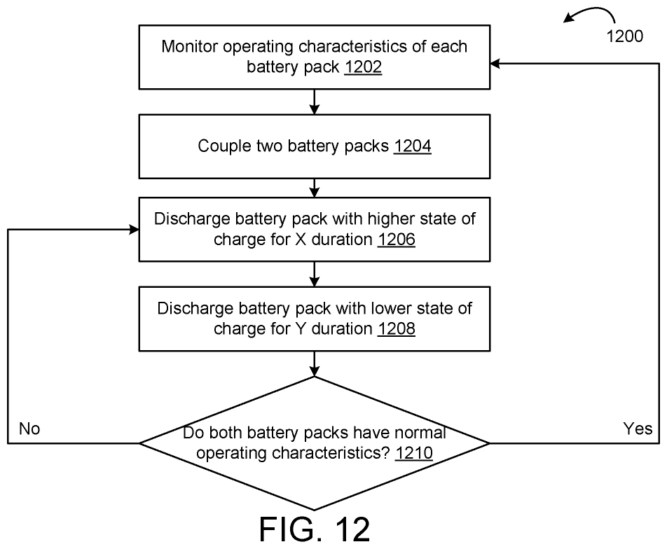

Referring to FIG. 12, another method 1200 for discharging batteries is shown, according to an exemplary embodiment. The system controller 302 and the battery controller 310 may implement the method 1200, at least in part. The system controller 302 may display the voltage measurements in a user interface (e.g., user interface 330) wherein the voltage measurements may be included within the information from an operating characteristic sensor 312.

The method 1200 begins at step 1202 with the system controller 302 monitors the operating characteristics of each of the battery packs 112a, 112b, 112c. The battery controller 310 may send the operation characteristics from the battery pack 112 through the communications interface 318, to the system controller 302. The system controller 302 may display the voltage measurements in a user interface (e.g., user interface 330) wherein the voltage measurements may be included within the information from an operating characteristic sensor 312.

At step 1204, two battery packs 112 will be coupled to allow for an incremental discharge. The battery packs 112 may be selected based on the operating characteristics from the operating characteristic sensors 312 in the battery controller 310. The operating characteristics are used to determine if the battery is operating outside the normal conditions specified in the memory 316 of the battery controller 310. The battery packs 112 may be coupled through the load bus 124 to allow the battery packs 112 to discharge at different times. The remaining battery packs 112 will be separate from the coupled battery packs 12 selected to discharge.

At step 1206, the system controller 302 may discharge the battery pack 112 with the highest state of charge for a specified duration. For ease of description, the battery controller 310 may establish the specified duration based on the operating characteristics of the battery pack 112. The battery controller 302 may provide more significance to the state of charge of the battery pack 112 when establishing the duration of discharge. The battery controller 302 may adjust the rate of discharge to maintain the optimal operation characteristics of the battery pack 112. The specified duration of the battery pack 112 with the higher state of charge may be longer than the duration if the battery pack 112 with the lower state of charge.

At step 1208, the system controller 302 may discharge the battery pack 112 with the lowest state of charge for a specified duration. For ease of description, the battery controller 310 may establish the specified duration based on the operating characteristics of the battery pack 112. The battery controller 302 may provide more significance to the state of charge of the battery pack 112 when establishing the duration of discharge. The battery controller 302 may adjust the rate of discharge to maintain the optimal operation characteristics of the battery pack 112. The specified duration of the battery pack 112 with the lower state of charge may be longer than the duration if the battery pack 112 with the higher state of charge.

At step 1210, the battery controller 310 will monitor the operating characteristics of both battery packs 112, while both are discharging at specified durations. If the battery controller detects that the operating characteristics are not normal, then the method 1200 returns again to step 1206. The battery packs may continue to discharge at a specified duration and rate. If the battery controller 310 detects that, the operating characteristics are normal, then the method 1200 returns to step 1202. The system controller 302 may decouple the battery packs 112 to maintain optimal characteristics. The method 1200 will continuously occur to protect the integrity of each battery pack 112.

Moreover, the system controller 302 alternates between connecting two battery packs 112. By way of example, the system controller 302 may alternate between (a) connecting a first battery pack 112 for a first duration while a second battery pack is disconnected and (b) connecting the second battery pack 112 for a second duration while the first battery pack is disconnected. The lengths of the first duration and the second duration (e.g., the ratio between the first duration and the second duration) may vary based on a state of charge or voltage of each battery pack 112. By way of example, the first duration may be greater than the second duration if the state of charge of the first battery pack 112 is greater than the state of charge of the second battery pack 112. In one example, the system controller 302 discharges a high voltage battery pack 112 for 6 seconds, then subsequently discharges a lower voltage battery pack 112 for 2 seconds, then repeats this alternating process over time.

### Configuration of the Exemplary Embodiments

The processors of the system (e.g., the processor 304 or the processor 314 etc.) may be one or more of a single-or multi-chip processor, a digital signal processor (DSP), an application specific integrated circuit (ASIC), a field programmable gate array (FPGA), or other programmable logic device, discrete gate or transistor logic, discrete hardware components, another type of suitable processor, or any combination thereof designed to perform the functions described herein. In this way, the processors may each be a microprocessor, a state machine, or other suitable processor. The processors also may be implemented as a combination of computing devices, such as a combination of a DSP and a microprocessor, a plurality of microprocessors, one or more microprocessors in conjunction with a DSP core, or any other such configuration. Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. All such variations are intended to fall within the scope of the present disclosure.

The memories (e.g., memory, memory unit, storage device), such as the memory 306 and the memory 316 may include one or more devices (e.g., RAM, ROM, Flash memory, hard disk storage) for storing data and/or computer code for completing or facilitating the various processes, layers and modules described in the present disclosure. The memories may be communicably coupled to the processors to provide computer code or instructions to the processors for executing at least some of the processes described herein. Moreover, the memories may be or include tangible, non-transient volatile memory or non-volatile memory. Accordingly, the memories may include database components, object code components, script components, or any other type of information structure for supporting the various activities and information structures described herein.

As utilized herein, the terms “approximately”, “about”, “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

It should be understood that while the use of words such as desirable or suitable utilized in the description above indicate that the feature so described may be more desirable, it nonetheless may not be necessary and embodiments lacking the same may be contemplated as within the scope of the invention, the scope being defined by the claims that follow. In reading the claims, it is intended that when words such as “a,” “an,” or “at least one” are used there is no intention to limit the claim to only one item unless specifically stated to the contrary in the claim.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like, as used herein, mean the joining of two members directly or indirectly to one another. Such joining may be stationary (e.g., permanent, etc.) or moveable (e.g., removable, releasable, etc.). Such joining may be achieved with the two members or the two members and any additional intermediate members being integrally formed as a single unitary body with one another or with the two members or the two members and any additional intermediate members being attached to one another.

References herein to the positions of elements (e.g., “top,” “bottom,” “above,” “below,” “between,” etc.) are merely used to describe the orientation of various elements in the figures. It should be noted that the orientation of various elements may differ according to other exemplary embodiments, and that such variations are intended to be encompassed by the present disclosure.

As used herein, the term “circuit” or “circuitry” may include hardware structured to execute the functions described herein. In some embodiments, each respective “circuit” may include machine-readable media for configuring the hardware to execute the functions described herein. The circuit may be embodied as one or more circuitry components including, but not limited to, processing circuitry, network interfaces, peripheral devices, input devices, output devices, sensors, etc. In some embodiments, a circuit may take the form of one or more analog circuits, electronic circuits (e.g., integrated circuits (IC), discrete circuits, system on a chip (SOCs) circuits, etc.), telecommunication circuits, hybrid circuits, and any other type of “circuit.” In this regard, the “circuit” may include any type of component for accomplishing or facilitating achievement of the operations described herein. For example, a circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

The “circuit” may also include one or more processors communicably coupled to one or more memory or memory devices. In this regard, the one or more processors may execute instructions stored in the memory or may execute instructions otherwise accessible to the one or more processors. In some embodiments, the one or more processors may be embodied in various ways. The one or more processors may be constructed in a manner sufficient to perform at least the operations described herein. In some embodiments, the one or more processors may be shared by multiple circuits (e.g., circuit A and circuit B may comprise or otherwise share the same processor which, in some example embodiments, may execute instructions stored, or otherwise accessed, via different areas of memory). Alternatively, or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be implemented as one or more general-purpose processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively, or additionally, the one or more processors may be internal and/or local to the apparatus. In this regard, a given circuit or components thereof may be disposed locally (e.g., as part of a local server, a local computing system, etc.) or remotely (e.g., as part of a remote server such as a cloud based server). To that end, a “circuit” as described herein may include components that are distributed across one or more locations.

The construction and arrangement of the suspension as shown in the exemplary embodiments is illustrative only. Although only a few embodiments of the present disclosure have been described in detail, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements. It should be noted that the elements and/or assemblies of the components described herein may be constructed from any of a wide variety of materials that provide sufficient strength or durability, in any of a wide variety of colors, textures, and combinations. Accordingly, all such modifications are intended to be included within the scope of the present inventions. Other substitutions, modifications, changes, and omissions may be made in the design, operating conditions, and arrangement of the preferred and other exemplary embodiments without departing from scope of the present disclosure or from the spirit of the appended claims.

What is claimed is:

1. Power equipment comprising:
a chassis;
an implement coupled to the chassis; and
a battery system coupled to the chassis, the battery system comprising:
a first battery pack;
a second battery pack;
a circuit including a first switch configured to selectively couple the first battery pack to the implement and a second switch configured to selectively couple the second battery pack to the implement; and
a controller operatively coupled to the circuit and configured to:
control the first switch and the second switch according to a first control strategy in response to an indication that the power equipment is a first type of power equipment; and
control the first switch and the second switch according to a second control strategy in response to an indication that the power equipment is a second type of power equipment.

2. The power equipment of claim 1, wherein the first type of power equipment is a lawn mower, and wherein the second type of power equipment is a light tower.

3. A battery system for power equipment, the battery system comprising:
a first battery pack;
a second battery pack;
a circuit comprising at least one switch configured to selectively couple the first battery pack and the second battery pack to one another in parallel and selectively decouple the first battery pack and the second battery pack from one another; and
a controller operatively coupled to the circuit and configured to control the at least one switch to selectively couple and decouple the first battery pack and the second battery pack based on operating characteristics of the first battery pack and the second battery pack.

4. The battery system of claim 3, wherein the operating characteristics comprise a first voltage of the first battery pack and a second voltage of the second battery pack, wherein the controller is configured to:
determine whether a voltage difference between the first voltage and the second voltage is less than a threshold voltage;
operate the at least one switch to couple the first battery pack and the second battery pack to one another in parallel in response to a determination that the difference between the first voltage and the second voltage is less than the threshold voltage; and
allow the first voltage and the second voltage to equalize as a result of coupling the first battery pack and the second battery pack to one another in parallel.

5. The battery system of claim 3, wherein the operating characteristics comprise a first voltage of the first battery pack and a second voltage of the second battery pack, wherein the controller is configured to:
determine whether a voltage difference between the first voltage and the second voltage is less than a threshold voltage;
operate the at least one switch to maintain the first battery pack and the second battery pack electrically decoupled from one another in response to a determination that the difference between the first voltage and the second voltage is not less than the threshold voltage; and
at least one of:
charge the first battery pack without charging or discharging the second battery pack while the first battery pack and the second battery pack are electrically decoupled from one another; or
discharge the first battery pack without charging or discharging the second battery pack while the first battery pack and the second battery pack are electrically decoupled from one another.

6. The battery system of claim 3, further comprising an electrical bus;
wherein the at least one switch is operable to electrically connect the first battery pack and the second battery pack to the electrical bus in parallel with one another and electrically disconnect the first battery pack and the second battery pack from the electrical bus;
wherein the controller is configured to:
operate the at least one switch to electrically connect the first battery pack to the electrical bus while the second battery pack is electrically disconnected from the electrical bus;
determine whether a first predetermined condition is satisfied when the first battery pack is electrically connected to the electrical bus and the second battery pack is electrically disconnected from the electrical bus; and
operate the at least one switch to electrically connect the second battery pack to the electrical bus in parallel with the first battery pack in response to determining that the first predetermined condition is satisfied.

7. The battery system of claim 3, further comprising an electrical bus;
wherein the at least one switch is operable to electrically connect the first battery pack and the second battery pack to the electrical bus in parallel with one another and electrically disconnect the first battery pack and the second battery pack from the electrical bus;
wherein the controller is configured to:
operate the at least one switch to electrically connect the first battery pack and the second battery pack to the electrical bus in parallel with one another;
determine whether a second predetermined condition is satisfied when the first battery pack and the second battery pack to the electrical bus in parallel with one another; and
operate the at least one switch to electrically disconnect the second battery pack from the electrical bus in response to determining that the second predetermined condition is satisfied.

8. The battery system of claim 3, wherein the operating characteristics comprise at least one of a state of charge or a voltage of the first battery pack, wherein the controller is configured to:
operate the at least one switch to electrically connect the first battery pack to one or more loads while the second battery pack is electrically disconnected from the one or more loads, thereby causing the first battery pack to discharge while powering the one or more loads;
monitor the state of charge or the voltage of the first battery pack while the first battery pack is electrically connected to the one or more loads and the second battery pack is electrically disconnected from the one or more loads; and
in response to a determination that the state of charge or the voltage of the first battery pack has fallen below a threshold state of charge or voltage, operate the at least one switch to electrically disconnect the first battery pack from the one or more loads and electrically connect the second battery pack to the one or more loads, thereby causing the second battery pack to discharge while powering the one or more loads.

9. The battery system of claim 3, wherein the operating characteristics comprise a first voltage of the first battery pack and a second voltage of the second battery pack, wherein the controller is configured to:
operate the at least one switch to electrically connect the first battery pack to one or more loads while the second battery pack is electrically disconnected from the one or more loads, thereby causing the first battery pack to discharge while powering the one or more loads;
monitor the first voltage of the first battery pack and the second voltage of the second battery pack while the first battery pack is electrically connected to the one or more loads and the second battery pack is electrically disconnected from the one or more loads; and
in response to a determination that a difference between the first voltage and the second voltage is less than or equal to a threshold voltage difference, operate the at least one switch to electrically connect the second battery pack to the one or more loads in parallel with the first battery pack, thereby causing both the first battery pack and the second battery pack to discharge while powering the one or more loads.

10. The battery system of claim 3, wherein the operating characteristics comprise at least one of a voltage, a temperature, or an electric current of the first battery pack, wherein the controller is configured to:
operate the at least one switch to electrically connect the first battery pack to one or more loads, thereby causing the first battery pack to discharge while powering the one or more loads;
monitor the operating characteristics of the first battery pack while discharging the first battery pack to power the one or more loads; and
operate the at least one switch to electrically disconnect the first battery pack from one or more loads in response to a determination that at least one of:
the voltage of the first battery pack has fallen below a threshold voltage;
the temperature of the first battery pack has risen above a threshold temperature; or
the electric current of the first battery pack has risen above a threshold electric current.

11. The battery system of claim 3, wherein the operating characteristics comprise at least one a state of charge or a voltage of the first battery pack and the second battery pack, wherein the controller is configured to:
operate the at least one switch to alternate between:
a first state in which the first battery pack is electrically connected to one or more loads while the second battery pack is electrically disconnected from the one or more loads; and
a second state in which the first battery pack is electrically disconnected from the one or more loads while the second battery pack is electrically connected to the one or more loads;
determine amounts of time to remain in the first state and the second state based on the state of charge or the voltage of the first battery pack relative to the state of charge or the voltage of the second battery pack; and
operate the at least one switch to transition between the first state and the second state based on the amounts of time.

12. A method for operating a battery system for power equipment, the method comprising:
providing a circuit comprising at least one switch operable to selectively couple the first battery pack and the second battery pack to one another in parallel and selectively decouple the first battery pack and the second battery pack from one another;
monitoring operating characteristics of a first battery pack and a second battery pack;
controlling the at least one switch to selectively couple and decouple the first battery pack and the second battery pack based on the operating characteristics of the first battery pack and the second battery pack.

13. The method of claim 12, wherein the operating characteristics comprise a first voltage of the first battery pack and a second voltage of the second battery pack, the method comprising:
determining whether a voltage difference between the first voltage and the second voltage is less than a threshold voltage;
operating the at least one switch to couple the first battery pack and the second battery pack to one another in parallel in response to a determination that the difference between the first voltage and the second voltage is less than the threshold voltage; and
allowing the first voltage and the second voltage to equalize as a result of coupling the first battery pack and the second battery pack to one another in parallel.

14. The method of claim 12, wherein the operating characteristics comprise a first voltage of the first battery pack and a second voltage of the second battery pack, the method comprising:
determining whether a voltage difference between the first voltage and the second voltage is less than a threshold voltage;
operating the at least one switch to maintain the first battery pack and the second battery pack electrically decoupled from one another in response to a determination that the difference between the first voltage and the second voltage is not less than the threshold voltage; and
at least one of:
charging the first battery pack without charging or discharging the second battery pack while the first battery pack and the second battery pack are electrically decoupled from one another; or
discharging the first battery pack without charging or discharging the second battery pack while the first battery pack and the second battery pack are electrically decoupled from one another.

15. The method of claim 12, comprising:
operating the at least one switch to electrically connect the first battery pack to an electrical bus while the second battery pack is electrically disconnected from the electrical bus;
determining whether a first predetermined condition is satisfied when the first battery pack is electrically connected to the electrical bus and the second battery pack is electrically disconnected from the electrical bus; and
operating the at least one switch to electrically connect the second battery pack to the electrical bus in parallel with the first battery pack in response to determining that the first predetermined condition is satisfied.

16. The method of claim 12, comprising:
operating the at least one switch to electrically connect the first battery pack and the second battery pack to an electrical bus in parallel with one another;
determining whether a second predetermined condition is satisfied when the first battery pack and the second battery pack to the electrical bus in parallel with one another; and
operating the at least one switch to electrically disconnect the second battery pack from the electrical bus in response to determining that the second predetermined condition is satisfied.

17. The method of claim 12, wherein the operating characteristics comprise at least one of a state of charge or a voltage of the first battery pack, the method comprising:
operating the at least one switch to electrically connect the first battery pack to one or more loads while the second battery pack is electrically disconnected from the one or more loads, thereby causing the first battery pack to discharge while powering the one or more loads;
monitoring the state of charge or the voltage of the first battery pack while the first battery pack is electrically connected to the one or more loads and the second battery pack is electrically disconnected from the one or more loads; and
in response to a determination that the state of charge or the voltage of the first battery pack has fallen below a threshold state of charge or voltage, operating the at least one switch to electrically disconnect the first battery pack from the one or more loads and electrically connect the second battery pack to the one or more loads, thereby causing the second battery pack to discharge while powering the one or more loads.

18. The method of claim 12, wherein the operating characteristics comprise a first voltage of the first battery pack and a second voltage of the second battery pack, the method comprising:
operating the at least one switch to electrically connect the first battery pack to one or more loads while the second battery pack is electrically disconnected from the one or more loads, thereby causing the first battery pack to discharge while powering the one or more loads;
monitoring the first voltage of the first battery pack and the second voltage of the second battery pack while the first battery pack is electrically connected to the one or more loads and the second battery pack is electrically disconnected from the one or more loads; and
in response to a determination that a difference between the first voltage and the second voltage is less than or equal to a threshold voltage difference, operating the at least one switch to electrically connect the second battery pack to the one or more loads in parallel with the first battery pack, thereby causing both the first battery pack and the second battery pack to discharge while powering the one or more loads.

19. The method of claim 12, wherein the operating characteristics comprise at least one of a voltage, a temperature, or an electric current of the first battery pack, the method comprising:
operating the at least one switch to electrically connect the first battery pack to one or more loads, thereby causing the first battery pack to discharge while powering the one or more loads;
monitoring the operating characteristics of the first battery pack while discharging the first battery pack to power the one or more loads; and
operating the at least one switch to electrically disconnect the first battery pack from one or more loads in response to a determination that at least one of:
the voltage of the first battery pack has fallen below a threshold voltage;
the temperature of the first battery pack has risen above a threshold temperature; or
the electric current of the first battery pack has risen above a threshold electric current.

20. The method of claim 12, wherein the operating characteristics comprise at least one a state of charge or a voltage of the first battery pack and the second battery pack, the method comprising:
operating the at least one switch to alternate between:
a first state in which the first battery pack is electrically connected to one or more loads while the second battery pack is electrically disconnected from the one or more loads; and
a second state in which the first battery pack is electrically disconnected from the one or more loads while the second battery pack is electrically connected to the one or more loads;
determining amounts of time to remain in the first state and the second state based on the state of charge or the voltage of the first battery pack relative to the state of charge or the voltage of the second battery pack; and
operating the at least one switch to transition between the first state and the second state based on the amounts of time.
